(19) United States
(12) Patent Application Publication
(10) Pub. No.: US 2004/0248877 A1
(43) Pub. Date: Dec. 9, 2004

(54) Polycyclic diazodioxide-based Bcl-2 protein antagonists and use thereof

(76) Inventors: Sandeep Gupta, Concord, OH (US); David Michael Carrig, Painesville, OH (US); Raghavan Rajagopalan, Solon, OH (US); Prabhavathi Fernandes, Lawrenceville, NJ (US)

Correspondence Address: FISH & RICHARDSON PC, 225 FRANKLIN ST, BOSTON, MA, 02110, US

(21) Appl. No.: 10/837,220

(22) Filed: Apr. 30, 2004

Related U.S. Application Data
(60) Provisional application No. 60/466,344, filed on Apr. 30, 2003.

(51) Int. Cl. A61K 31/551; A61K 31/553

(52) U.S. Cl. 514/211.08; 514/219; 540/545; 540/555

(57) ABSTRACT

Compounds of Formula 8 are provided:

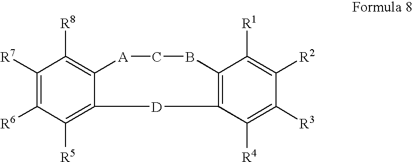

A and B are each independently selected from the group consisting of —NO—, —SO—, and —NR⁹—. C is a single bond or a double bond. D is selected from the group consisting of single bond,

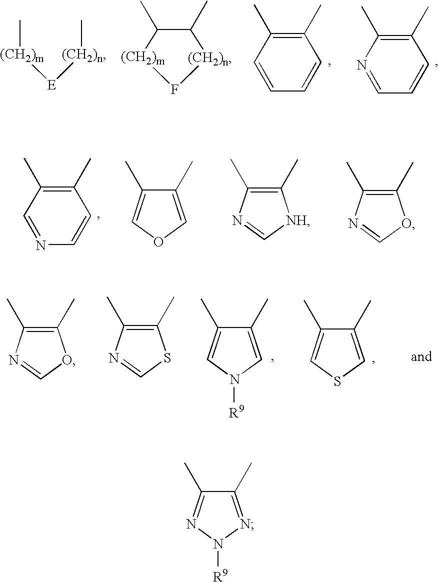

E is selected from the group consisting of single bond, double bond, —NR⁹—, —O—, —S—, —SO—, and —SO₂—; and m and n are each independently an integer from 0 to 6. R¹ to R⁹ are appropriately selected to optimize physicochemical and/or biological properties such as lipophilicity, bioavailability, pharmacokinetics, Bcl-2 and Bcl-X_{L} activities, metabolism, and the like. R¹ and R², R² and R³, R³ and R⁴, R⁵ and R⁶, R⁶ and R⁷, or R⁷ and R⁸ may optionally be joined together to form an aromatic or heteroaromatic ring, including, but not limited to, naphthyl, quinolinyl, isoquinolinyl, benzimidazolyl, benzoxazolyl, benzothiazolyl and the like. The compounds are useful for tumor therapeutic applications. These compounds induce apoptosis in tumor cells mediated through Bcl-2 family of proteins.

### RELATED APPLICATIONS

Priority is claimed herein to U.S. provisional patent application No. 60/466,344, to Gupta et al., filed Apr. 30, 2003, entitled “Novel Polycyclic Diazodioxide based Bcl-2 Protein Antagonists and the Use Thereof.” The disclosure of the above-referenced application is incorporated by reference herein in its entirety.

### FIELD

Provided herein are compositions for cancer therapy. More particularly, provided herein are polycyclic N-oxide and S-oxide derivatives and their use in cancer therapy mediated by the Bcl-2 family of proteins.

### BACKGROUND

One of the most promising, but challenging approaches for the treatment of cancer involves the selective induction of apoptosis (controlled cell death and disposal) in tumor cells. Apoptosis plays an important role not only in normal cell growth and maintenance, but also in defending the organism against pathogenic microorganisms. Also, multicellular organisms also use apoptotic process to destroy damaged DNA before it induces cancerous transformation. It is well recognized that one of the causes of cancer is the perturbation of the intricate balance (homeostasis) between growth and death, and that faulty regulation of apoptotic process has been implicated in many diseases including cancer, degenerative disorders and vascular diseases. See, e.g., Gross, A. et al. Genes Devel 1991, 13, 1899-1911; Hawkins, C. J.; Vaux, D. L. Semin. Immunol. 1997, 9, 25-33. There are two major challenges in treating tumors using apoptotic process: first, many cancer cells have a mechanism to evade the apoptotic process; and second, the treatment protocol requires delicate balance between growth and death of normal versus cancerous cells, for too much activation of apoptotic process will result in death of normal cells and too little activation or inactivation may cause proliferation of cancer cells.

Over the past several decades, many apoptotic regulators have been identified, which include Bcl-2 (B-cell lymphoma) family of proteins. The Bcl-2 family comprises both anti-apoptotic proteins such as Bcl-2 itself, Bcl-X_{L}, Bcl-w, Mcl, and A1; and pro-apoptotic proteins such as Bax, Bak, Bad, Bik, Bid, and Bok (Adams, J. M. and Cory, S. Science 1998, 281, 1322-1326). The direct link of the BCL2 gene to apoptosis and cancer emerged when this key gene in follicular lymphoma was found to inhibit cell death rather than promote proliferation.

In humans, 24 members of the Bcl-2 group of proteins have been identified. These proteins are the central regulators of the intrinsic apoptotic pathway and they regulate integrity of mitochondrial membrane. Changes in the permeability or destruction of the mitochondrial membranes leads to the release of cytochrome-C and other apoptotic proteins that in concert with apoptotic protein activating factor, Apaf1, carry out the activation of the initiator caspase 9. Caspases are a key group of intracellular cysteine activated-aspartate specific proteases (11 members identified in humans) which are present as inactive precursors-but upon activation, produce a cascade of proteolytic events leading to cell death. Once the initiator caspase is activated, it processes others that begin to degrade a multitude of cellular proteins signaling the initiation of the apoptotic process.

Bcl-2 itself is a 26 kilodalton protein and is related to the other members of the group by the presence of highly conserved homology domains (BH1-BH4). The pro-survival group of proteins such as Bcl-2 and Bcl-X_{L} carry all the BH1-BH4 homology domains while the Bax family of pro-apoptotic proteins are characterized by the presence of BH1-BH3. The last group of proteins, the BH3 only proteins such as Bid, Bim, Bik, and Bad, are considered to be sentinel proteins responsible for triggering apoptosis in response to apoptotic signal. Both Bcl-2 and Bcl-X_{L} are over-expressed in several type of tumors, including 70% of breast cancers, 80% of B-cell lymphomas, 30-60% of prostate cancers, and 90% of colorectal adenocarcinomas (Buolamvini, J. K. Curr. Opin. Chem. Biol. 1991, 3, 500-509). Overexpression of Bcl-2 and Bcl-X_{L} results in blocking of apoptotic signals that leads to cell proliferation. Although the precise mechanism of action of Bcl-2 is not clearly understood, it is believed that the anti-apoptotic Bcl-2 proteins prevent the release of pro-apoptotic factors such as cytochrome-C from mitrochondria, thereby inhibiting the initiation of activities of a group of proteolytic enzymes that actually causes cell destruction (Kelekar, A. and Thompson, B. Trends Cell Biol. 1998, 8, 324-330). It has also been shown that Bcl-2 is a mitochondrial membrane-bound protein that maintains the integrity of mitrochondria and its dissociation from the membrane causes degradation of mitochondiral membrane and thereby releasing proteolytic enzyme from the mitochondrion (Cory, S. and Adams, J. M. Nature Reviews, Cancer 2002, 2, 647). The levels of Bcl-2 proteins have been shown to correlate with the resistance to many chemotherapeutic drugs and radiation therapy, and that the suppression of their activity and/or their levels restores the sensitivity to the aforementioned therapeutic agents (Reed, J. C. Adv. Pharmacol. 1997, 41, 501-553). In essence, conventional cytotoxic therapy indirectly induces apoptosis through the intrinsic pathway, but cancer cells often show diminished response to such therapies. A better response, however, can be elicited by direct induction of apoptosis using processes such as impairing the action or expression of Bcl-2 like proteins or identifying compounds that mimic the BH3 only proteins.

The activities of Bcl-2 and Bcl-X_{L} are intimately connected to their binding at the BH3 region of the pro-apoptotic proteins Bax, Bak, Bid, and Bad, and the formation of such heterodimeric complex between pro- and anti-apoptotic proteins has been shown to induce apoptosis and suppression of tumor growth in animal model systems (Wang, J. L. et al. Proc. Natl. Acad. Sci. U.S.A. 2000, 97, 7124-7129). There is ample evidence in the form of NMR and X-ray data supporting the protein-protein interaction leading to the formation of heterodimers. For example the data suggests that the Bak peptide in the complex adopts an amphipathic a helical structure (BH3 domain) that interacts with Bcl-X_{L} through hydrophobic and electrostatic interactions. Mutations in Bak that prevent these interactions inhibit the ability of Bak to form the heterodimer with Bcl-X_{L}. In essence, the pro-apoptotic protein sequesters the anti-apoptotic protein and, thus, it is reasonable to expect that the development of small molecule antagonists of Bcl-2 or Bcl-X_{L} present viable and attractive targets for cancer chemotherapy. Their natural survival function can be eliminated using strategies such as turning off the gene transcription, use of antisense oligonucleotides to inactivate mRNA, or directly modifying protein activity using small molecule therapeutics. Accordingly, there has been considerable effort in developing small molecules directed at not only perturbing the protein-protein interaction, but also inhibiting the gene expression of anti-apoptotic proteins (Enyedy, I. J. et al. J. Med. Chem. 2001, 44, 4313-4324; Wang, S. and Carroll, P. G. 2002, WO 02/097053 A2; Zeigler, A. et al. J. Natl. Cancer. Inst. 1997, 89, 1027-1036). These small molecules (Table 1) include both natural products such as gossypol (1) and antimycin (2) and synthetic compounds 3-6. The efficacy of a drug substance depends not only on the strength of the binding of these molecules to the cellular components, but also equally importantly on pharmacokinetic and pharmacodynamic parameters, including cell permeability, metabolism, serum protein binding, and the like. Most of the compounds screened thus far, including those listed in Table 1, exhibited only a modest activity, both with respect to Bcl-2 protein binding as well as cytotoxicity. Although compound 3 was disclosed as a potential anticancer compound in 1983 (Bown, D. Ph.D. Thesis 1983, Massachusetts Institute of Technology), its mode of action as Bcl-2 antagonist has been demonstrated only recently (Wang, S. and Carroll, P. G. 2002, WO 02/13833 A2). Furthermore, the structure-activity relationship (SAR) data is very limited despite its attractive feature of having a simple structure and moderate activity. Most recently, de novo design of Bcl-2 antagonists by molecular modeling method yielded a potent, but highly lipophilic compound 7 with an IC₅₀ value of 114 nM (Olaf, K. et al. J. Am. Chem. Soc. 2002, 124, 11838). However, its efficacy in cell-based assay has not been established. Thus, there continues to exist the need to develop novel small molecule compositions having optimal Bcl-2 and Bcl-X_{L} binding and pharmacological properties.

TABLE 1
Small Molecule Inhibitors of Bcl-2 and Bcl X_{L}

| Compound | | IC₅₀ (FP) | IC₅₀ (cell) | Reference |
|---|---|---|---|---|
| 1 | 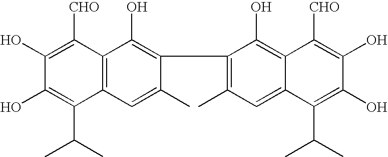 | 10 μM (Bcl-2) 0.4 μM (Bcl-X_{L}) | 1.5 μM | 10 |
| | Gossypol | | | |
| 2 | 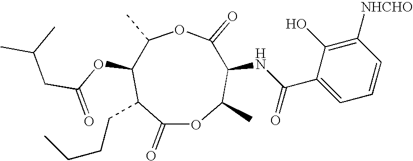 | 2.5 μM | 1.2 μM | 15 |
| | Antimycin | | | |
| 3 | 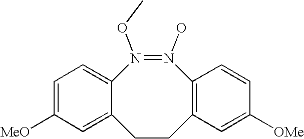 | 10 μM | 10 μM | 9, 13 |
| 4 | 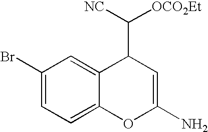 | 9 μM | 18 μM | 8, 16 |
| 5 | 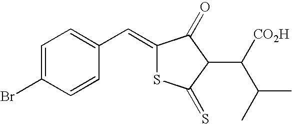 | 2.4 μM | ˜90 μM | 17 |
| 6 | 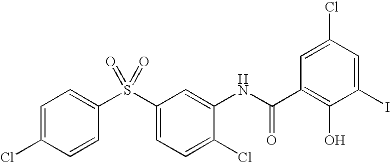 | 3.3 μM | ˜30 μM | 17 |
| 7 | 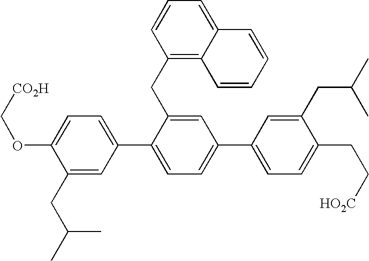 | 114 nm | Not Reported | 14 |

### SUMMARY

Provided herein are compounds and pharmaceutical compositions containing compounds having Formula 8:

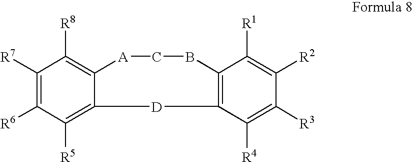

or a pharmaceutically acceptable derivative thereof, where A and B are each independently selected from the group consisting of —NO—, —SO—, —SO₂—, and —NR⁹—. C is a single bond or a double bond. D is selected from the group consisting of single bond,

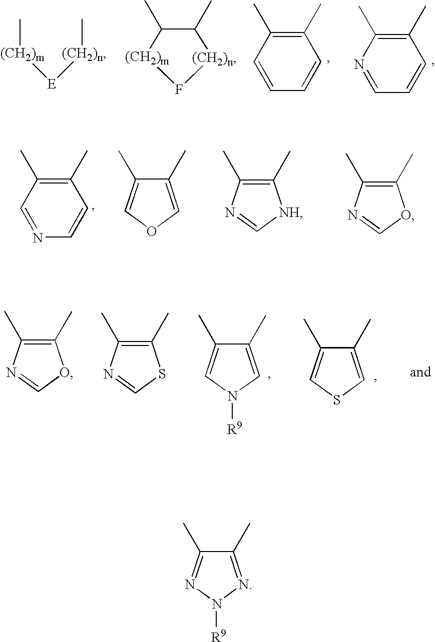

E and F are each independently selected from the group consisting of single bond, double bond, —NR⁹—, —O—, —S—, —SO—, and —SO₂—; and m and n are each independently an integer from 0 to 6. In one embodiment, R¹ to R⁹ are appropriately selected to optimize physicochemical and/or biological properties such as lipophilicity, bioavailability, pharmacokinetics, Bcl-2 and Bcl-X_{L} activities, metabolism, and the like. In certain embodiments, the compounds are selected with the proviso that if E is a single bond, A and B are both —NO— and m and n are both 1, then R³ and R⁶ both are not hydrogen, hydroxyl, halo, alkoxyl, alkenyloxyl, cycloalkoxyl, phenoxyl, or trifluoromethoxyl and R² and R⁷ both are not methyl, halo, and methoxycarbonyl. In another embodiment, R¹ and R², R² and R³, R³ and R⁴, R¹ and R⁶, R⁶ and R⁷, or R⁷ and R⁸ may optionally be joined together to form an aromatic or heteroaromatic ring including, but not limited to, naphthyl, quinolinyl, isoquinolinyl, benzimidazolyl, benzoxazolyl, benzothiazolyl and the like.

Also of interest are any pharmaceutically-acceptable derivatives, including salts, esters, enol ethers or esters, acetals, ketals, orthoesters, hemiacetals, hemiketals, solvates, hydrates or prodrugs of the compounds. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-parachlorobenzyl-2-pyrrolidin-1′-ylmethylbenzimidazole, diethylamine and other alkylamines, piperazine, tris(hydroxymethyl)aminomethane, alkali metal salts, such as but not limited to lithium, potassium and sodium, alkali earth metal salts, such as but not limited to barium, calcium and magnesium, transition metal salts, such as but not limited to zinc and other metal salts, such as but not limited to sodium hydrogen phosphate and disodium phosphate, and also including, but not limited to, salts of mineral acids, such as but not limited to hydrochlorides and sulfates, salts of organic acids, such as but not limited to acetates, lactates, malates, tartrates, citrates, ascorbates, succinates, butyrates, valerates and fumarates.

Pharmaceutical formulations for administration by an appropriate route and means containing effective concentrations of one or more of the compounds provided herein or pharmaceutically acceptable derivatives, such as salts, esters, enol ethers or esters, acetals, ketals, ortho esters, hemiacetals, hemiketals, solvates, hydrates or pro drugs, of the compounds that deliver amounts effective for the treatment of Bcl-2 protein-mediated disorders, are also provided. Bcl-2 protein-mediated disorders include, but are not limited to, cancers, tumors, hyperproliferative diseases, acquired immune deficiency syndrome, degenerative conditions, and vascular diseases. In certain embodiments, the cancers include, but are not limited to B-cell lymphoma including B-cell lymphoma-2, B-cell leukemia, skin cancer, pancreatic cancer, ovarian cancer, liver cancer, bladder cancer, adrenal carcinoma, breast cancer, prostate cancer, colorectal cancer including colorectal adenocarcinomas, follicular lymphoma.

The formulations are compositions suitable for administration by any desired route and include solutions, suspensions, emulsions, tablets, dispersible tablets, pills, capsules, powders, dry powders for inhalation, sustained release formulations, aerosols for nasal and respiratory delivery, patches for transdermal delivery and any other suitable route. The compositions should be suitable for oral administration, parenteral administration by injection, including subcutaneously, intramuscularly or intravenously as an injectable aqueous or oily solution or emulsion, transdermal administration and other selected routes.

Methods using such compounds and compositions for modulating the activity of a Bcl-2 protein are provided. The methods are effected by contacting a composition containing the Bcl-2 protein with one or more of the compounds or compositions.

Methods for treatment of Bcl-2 protein-mediated disorders, including, but not limited to, Bcl-2 protein-mediated disorders include, but are not limited to, cancers, tumors, hyperproliferative diseases, acquired immune deficiency syndrome, degenerative conditions, and vascular diseases. In certain embodiments, the cancers include, but are not limited to B-cell lymphoma including B-cell lymphoma-2, B-cell leukemia, skin cancer, pancreatic cancer, ovarian cancer, liver cancer, bladder cancer, adrenal carcinoma, breast cancer, prostate cancer, colorectal cancer including colorectal adenocarcinomas, follicular lymphoma.

In practicing the methods, effective amounts of formulations containing therapeutically effective concentrations of the compounds formulated for oral, intravenous, local and topical application for the treatment of Bcl-2 protein-mediated diseases or disorders are administered to an individual exhibiting the symptoms of one or more of these disorders. The amounts are effective to ameliorate or eliminate one or more symptoms of the diseases or disorders.

Articles of manufacture containing packaging material, a compound provided herein, or a pharmaceutically acceptable derivative thereof, which is effective for ameliorating the symptoms of a Bcl-2 protein-mediated disorder, within the packaging material, and a label that indicates that the compound, or pharmaceutically acceptable derivative thereof, is used for ameliorating the symptoms of a Bcl-2 protein-mediated disorder are provided.

### DETAILED DESCRIPTION

A. Definitions

Unless defined otherwise, all technical and scientific terms used herein have the same meaning as is commonly understood by one of ordinary skill in the art to which the claimed subject matter belongs. All patents, applications, published applications and other publications are incorporated by reference in their entirety. In the event that there are a plurality of definitions for a term herein, those in this section prevail unless stated otherwise.

As used herein, a “Bcl-2 protein” is a member of a class of proteins affecting apoptosis. The class includes at least 24 individual proteins. The proteins are both pro-apoptotic (e.g., Bax, Bak, Bad, Bik, Bid and Bok) and anti-apoptotic (e.g., Bcl-2, Bcl-X_{L}, Bcl-w, Mcl and A1).

As used herein, “Bcl-2” refers to the specific protein designated Bcl-2.

As used herein, an “anti-apoptotic Bcl-2 protein” is a Bcl-2 protein whose activity prevents or delays apoptosis. Such proteins include but are not limited to Bcl-2, Bcl-XL, Bcl-w, Mcl and A1.

As used herein, a “pro-apoptotic Bcl-2 protein” is a Bcl-2 protein whose activity induces or assists apoptosis. Such proteins include but are not limited to Bax, Bak, Bad, Bik, Bid and Bok.

As used herein, pharmaceutically acceptable derivatives of a compound include salts, esters, enol ethers, enol esters, acetals, ketals, orthoesters, hemiacetals, hemiketals, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-parachlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating Bcl-2 protein mediated diseases or disorders, or diseases or disorders in which Bcl-2 protein activity is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, LC₅₀ refers to a concentration of a particular test compound that kills 50% of cells in an in vitro assay that measures such response, including the assays described herein.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers.

Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, alkyl, alkenyl and alkynyl carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl(propenyl) and propargyl(propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are each independently alkyl, including lower alkyl; “carboxamide” refers to groups of formula —NR′COR in which R′ and R are each independently alkyl, including lower alkyl.

As used herein, “diarylaminocarbonyl” refers to —C(O)NRR′ in which R and R′ are each independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ—, where n and m are each independently an integer from 0 to 4.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—H═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein, in one embodiment selected from Q¹.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylidene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens. As another example, “C₁₋₃alkoxyphenyl” may include one or more of the same or different alkoxy groups containing one, two or three carbons.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Compounds

Provided herein are compounds and pharmaceutical compositions containing compounds of Formula 8:

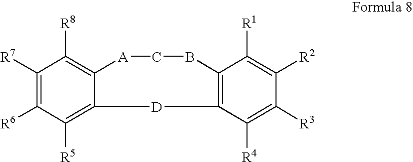

or a pharmaceutically acceptable derivative thereof, where A and B are each independently selected from the group consisting of —N—, —NO—, —SO—, —SO₂—, and —NR⁹—; C is a single bond or a double bond; D is selected from the group consisting of single bond,

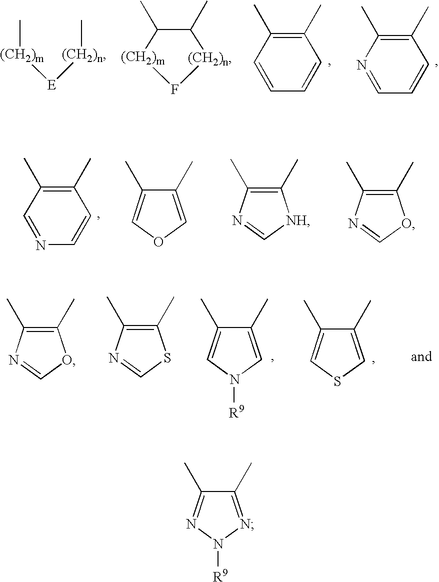

E and F are each independently selected from the group consisting of a single bond, double bond, —NR⁹—, —CR¹⁰R¹¹, —O—, —S—, —SO—, and —SO₂—; m and n are each independently an integer from 0 to 6; R¹ to R⁸, R¹⁰, and R¹¹ are each independently selected from the group consisting of hydrogen, C1-C10 alkyl, C3-C10 cycloalkyl, C1-C10 acyl, C5-C10 aryl, C1-C10 alkoxy, C1-C10 alkoxyalkyl, C1-C10 aralkoxy, C1-C10 heteroaralkoxy, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, C1-C10 alkylcarbonylamino, C1-C10 mono- or polyhaloalkylcarbonylamino, halo, mono- or polyhaloalkyl, mono- or polyhaloalkoxyl, cyano, nitro, mercapto, C1-C10 mercaptoalkyl, C1-C10 thioalkyl, C1-C10 alkylthioalkyl, C1-C10 sulfonylalkyl, and C1-C10 alkylsulfonylalkyl; and R⁹ is selected from the group consisting of hydrogen, C1-C10 alkyl, C3-C10 cycloalkyl, C1-C10 acyl, C5-C10 aryl, C1-C10 alkoxyalkyl, C1-C10 aminoalkyl, C1-C10 alkylaminoalkyl, hydroxyl, C1-C10 hydroxyalkyl, C1-C10 carboxyalkyl, C1-C10 alkoxylcarbonyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl.

In certain embodiments, the compounds are selected with the proviso that if E is a single bond, A and B are both —NO— and m and n are both 1, then R³ and R⁶ both are not hydrogen, hydroxyl, halo, alkoxyl, alkenyloxyl, cycloalkoxyl, phenoxyl, or trifluoromethoxyl and R² and R⁷ both are not methyl, halo, and methoxycarbonyl.

In other embodiments, the compounds are selected with the proviso that if E is a single bond, A and B are both —NO and m and n are both 1, then R³ and R⁶ both are not hydrogen, hydroxyl, halo, alkoxyl, phenoxyl, or trifluoromethoxyl and R² and R⁷ both are not methyl, halo, and methoxycarbonyl.

In certain embodiments, the compounds are selected with the proviso that if E is a single bond, A and B are both —NO— and m and n are both 1, then R³ and R⁶ both are not hydrogen, hydroxyl, alkoxyl, or trifluoromethoxyl and R² and R⁷ both are not methyl.

In other embodiment, the compounds are selected with the proviso that if E is a single bond and m and n are both 1, then R³ and R⁶ both are not hydrogen, alkoxyl, or trifluoromethoxyl and R² and R⁷ both are not methyl.

In another embodiment, R¹ to R⁸, R¹⁰, and R¹¹ are each independently selected from the group consisting of hydrogen, C1-C10 alkyl, C3-C10 cycloalkyl, C1-C10 acyl, C5-C10 aryl, C1-C10 alkoxyl, C1-C10 alkoxyalkyl, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, halo, mono- or polyhaloalkyl, mono- or polyhaloalkoxyl, cyano, nitro, mercapto, C1-C10 mercaptoalkyl, C1-C10 thioalkyl, C1-C10 alkylthioalkyl, C1-C10 sulfonylalkyl, and C1-C10 alkylsulfonylalkyl.

In another embodiment, the compounds provided herein have Formula 8, or a pharmaceutically acceptable derivative thereof, where A and B are each independently —NO— or —N—; C is a double bond; D is selected from the group consisting of single bond,

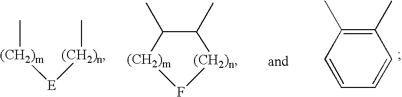

E and F are each independently selected from the group consisting of single bond, double bond, —NR⁹—, —CR¹⁰R¹¹—, and —O—; and m and n each independently an integer from 0 to 6; R¹ to R⁸, R¹⁰, and R¹¹ are each independently selected from the group consisting of hydrogen, C1-C10 alkyl, C5-C10 aryl, C1-C10 alkoxyl, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, halogen, mono- or polyfluroalkyl, mono- or polyfluroalkoxyl, cyano, nitro, C1-C10 thioalkyl, C1-C10 sulfonylalkyl, and C1-C10 alkylsulfonylalkyl; and R⁹ is selected from the group consisting of hydrogen, C1-C10 alkyl, C3-C10 cycloalkyl, C1-C10 acyl, C5-C10 aryl, C1-C10 alkoxyalkyl, C1-C10 hydroxyalkyl, C1-C10 carboxyalkyl, C1-C10 alkoxylcarbonyl, and C1-C10 alkoxycarbonylalkyl.

In another embodiment, the compounds provided herein have Formula 8, where A and B are —NO—; C is a double bond; D is selected from the group consisting of single bond,

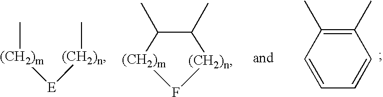

E and F are each independently single bond, double bond, —NR⁹, or —O—; and m and n each independently an integer from 0 to 6; R¹ to R⁸, R¹⁰, and R¹¹ are each independently selected from the group consisting of hydrogen, C1-C10 alkyl, C5-C10 aryl, C1-C10 alkoxyl, amino, hydroxyl, C1-C10 hydroxyalkyl, carboxyl, C1-C10 alkoxylcarbonyl, mono- or polyfluroalkyl, mono- or polyfluroalkoxyl, cyano, nitro, C1-C10 thioalkyl, and C1-C10 sulfonylalkyl; and R⁹ is selected from the group consisting of hydrogen, C1-C10 alkyl, C3-C10 cycloalkyl, C1-C10 acyl, C5-C10 aryl, carboxyalkyl, C1-C10 alkoxylcarbonyl, and C1-C10 alkoxycarbonylalkyl.

In another embodiment, the compounds provided herein have Formula 8, wherein A and B are —NO—; C is a double bond; D is selected from the group consisting of

single bond,

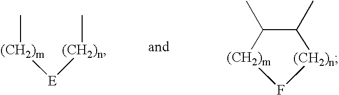

E and F are each independently single bond, double bond, —NR⁹—, or —O—; and m and n are each independently an integer from 0 to 6; R¹ to R⁸, R¹⁰, and R¹¹ are each independently selected from the group consisting of hydrogen, C1-C10 alkyl, C1-C10 alkoxyl, mono- or polyfluroalkyl, and mono- or polyfluroalkoxyl and R⁹ is selected from the group consisting of hydrogen, C1-C10 alkyl, C3-C10 cycloalkyl, C1-C10 acyl, and C5-C10 aryl, and C1-C10 alkoxylcarbonyl.

In another embodiment, the compounds provided herein have Formula 8, wherein A and B are —SO—; C is a single bond; D is selected from the group consisting of

single bond,

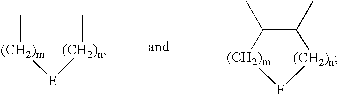

E and F are each independently single bond, double bond, —NR⁹—, or —O—; and m and n each independently an integer from 0 to 6; R¹ to R⁸, R¹⁰, and R¹¹ are each independently selected from the group consisting of hydrogen, C1-C10 alkyl, C1-C10 alkoxyl, mono- or polyfluroalkyl, and mono- or polyfluroalkoxyl; and R⁹ is selected from the group consisting of hydrogen, C1-C10 alkyl, C3-C10 cycloalkyl, C1-C10 acyl, and C5-C10 aryl, and C1-C10 alkoxylcarbonyl.

In another embodiment, at least one of R¹-R⁸, in another embodiment two of R¹-R⁸, are electron withdrawing substituents. Such substituents include but are not limited to haloalkoxy, alkylsulfonyl and haloamido groups.

In another embodiment, R¹, R⁴, R⁵ and R⁸ are each hydrogen.

In another embodiment, -A-C—B— is —NO═NO—.

In another embodiment, D is alkylene or a single bond. In another embodiment, D is ethylene or a single bond.

In another embodiment, R² and R⁷ are each independently hydrogen or C1-C10 polyhaloalkylcarbonylamino. In another embodiment, R² and R⁷ are each independently hydrogen or trifluoromethylcarbonylamino. In another embodiment, R² and R⁷ are each hydrogen. In another embodiment, R² and R⁷ are each trifluoromethylcarbonylamino.

In another embodiment, R³ and R⁶ are each independently hydrogen, polyhaloalkoxy, halo, aralkoxy, alkylsulfonyl or polyhaloalkylheteroaryloxy. In another embodiment, R³ and R⁶ are each independently hydrogen, difluoromethoxy, 2,2,2-trifluoroethoxy, 1,1,2-trifluoro-2-chloroethoxy, 1,1,2,3,3,3-hexafluoropropoxy, fluoro, benzyloxy, ethanesulfonyl or 5-trifluoromethyl-2-pyridyloxy. In another embodiment, R³ and R⁶ are each independently polyhaloalkoxy, halo, aralkoxy, alkylsulfonyl or polyhaloalkylheteroaryloxy. In another embodiment, R³ and R⁶ are each independently difluoromethoxy, 2,2,2-trifluoroethoxy, 1,1,2-trifluoro-2-chloroethoxy, 1,1,2,3,3,3-hexafluoropropoxy, fluoro, benzyloxy, ethanesulfonyl or 5-trifluoromethyl-2-pyridyloxy.

In certain embodiments, the compounds for use in the compositions and methods provided herein have formula 22:

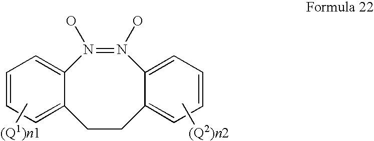

wherein n₁ and n₂ are each independently an integer from 1 to 4 and Q¹ and Q² are each independently selected from electron withdrawing substituents. Such substituents include but are not limited to haloalkoxy, alkylsulfonyl and haloamido groups.

In other embodiments, the compounds have formula 23:

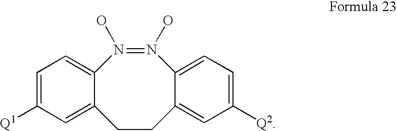

In certain embodiments, the compounds include tautomers of compounds provided herein, including ring opened tautomers of the compounds of formula 22 and formula 23.

In certain embodiments the compounds for use in the compositions and methods provided herein are selected from Table 2. Table 2 provides in vitro data in HL60, human leukemic cells expressing high Bcl-2 and low Bcl-X_{L} levels, and A549 human lung cells for exemplary compounds, calculated as described in example 3. Average LC₅₀ is provided as follows: a=<50 μM, b=50-200 μM, c>200 μM and n/c=data not calculated.

TABLE 2

LC 50 (μM)

| S. No. | Structure | A549 | HL-60 |
|---|---|---|---|
| 1 | 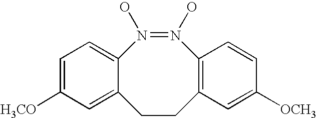 | n/c | b |
| 2 | 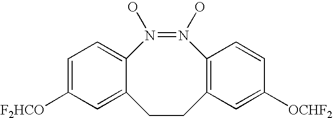 | c | a |
| 3 | 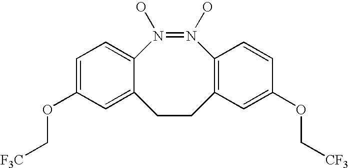 | b | a |
| 4 | 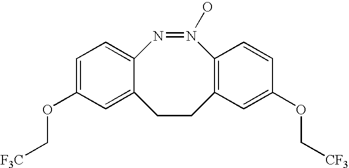 | c | c |
| 5 | 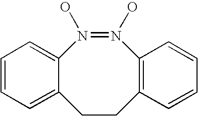 | c | c |
| 6 | 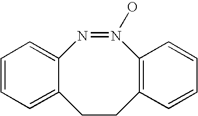 | c | c |
| 7 | 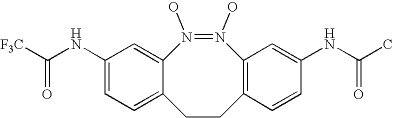 | c | c |
| 8 | 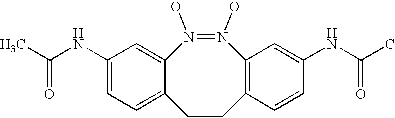 | c | c |
| 9 | 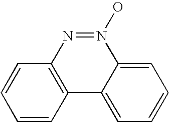 | c | c |
| 10 | 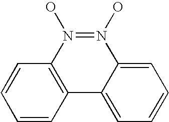 | c | c |
| 11 | 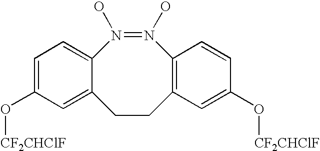 | c | a |
| 12 | 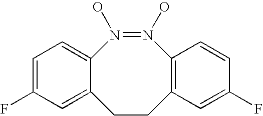 | c | c |
| 13 | 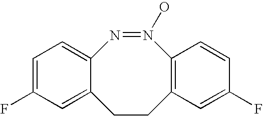 | c | c |
| 14 | 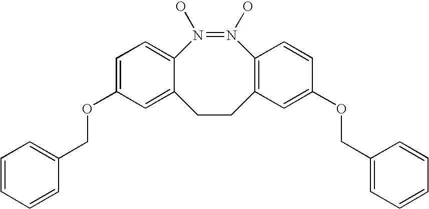 | c | c |
| 15 | 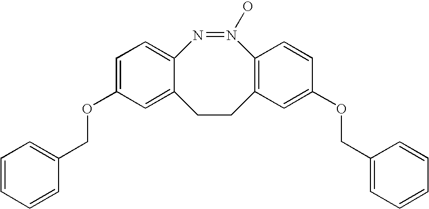 | c | c |
| 16 | 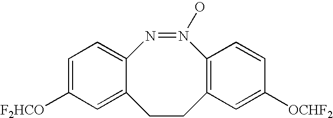 | c | c |
| 17 | 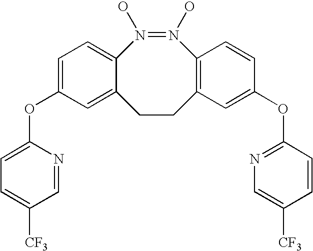 | c | b |
| 18 | 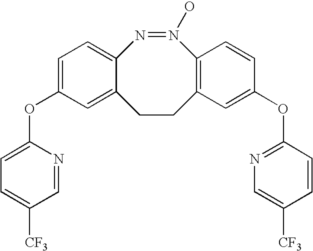 | c | c |
| 19 | 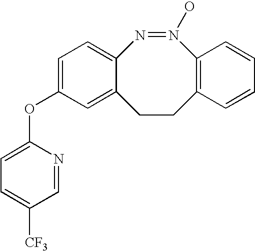 | c | c |
| 20 | 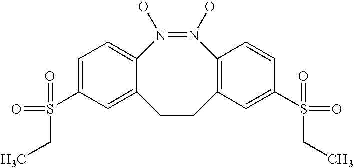 | c | c |
| 21 | 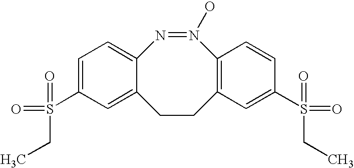 | c | c |
| 22 | 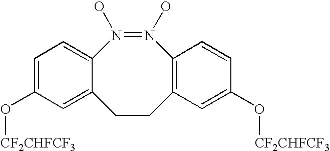 | c | a |

In another embodiment, the compounds for use in the compositions and methods provided herein are selected from:

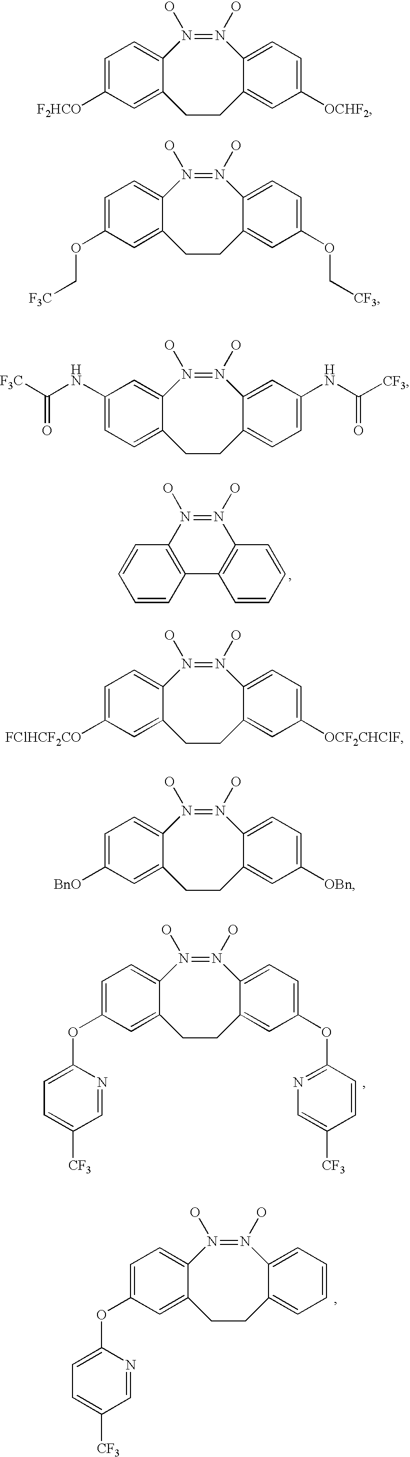

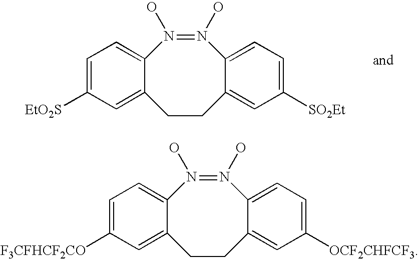

C. Preparation of the Compounds

The compounds belonging to Formula 8 can be prepared by standard synthetic methods known in the art. The examples that follow are not purported to limit the scope of the subject matter claimed herein. It is intended that the specification, together with the following examples, be considered exemplary only, with the scope and spirit of the subject matter disclosed herein being indicated by the claims that follow these examples. Other embodiments within the scope of claims herein will be apparent to one skilled in the art from consideration of the specification or practice of the subject matter described herein.

In one embodiment, compounds of Formula 8 wherein E is a double bond may be prepared in three steps as outlined in Scheme 1. The conditions to carryout the first step, viz., the Wittig reaction, are described in the literature, which is incorporated herein by reference in its entirety (Molina, P. et al. J. Org. Chem. 1996, 61, 4289). The reduction of the intermediate dinitro compound may be carried out by treatment with iron in acetic acid or ethanolic hydrochloric acid, or by hydrogenation using palladium on carbon or platinum oxide as catalyst. The reaction can also be carried out under conditions such as treatment with hydrazine hydrate in the presence of catalysts such as palladium on carbon or platinum oxide, or treatment with hydrazine in the presence of catalyst such as Raney nickel. The final oxidation step of the diamine to 11 may be carried out by the treatment with oxidants such as hydrogen peroxide, hydrogen peroxide-urea adduct, organic peracids such as meta chloroperoxybenzoic acid, or OXONE®. The reaction is conducted usually in the presence of a solvent, if necessary, in the presence of a catalyst such as sodium tungstate. The solvent may, for example, be an aromatic hydrocarbon such as benzene or toluene; an ether such as diethyl ether, tetrahydrofuran or dioxane; a halogenated hydrocarbon such as methylene chloride or chloroform; an aprotic polar solvent such as acetonitrile, dimethylformamide or pyridine or a protic polar solvent such as ethanol or methanol. The reaction temperature is usually from −50° to +150° C., In one embodiment, from −20° to 50° C. The reaction time is from 0.1 to 24 hours. Alternatively, cis olefin 11 can also be prepared by olefin metathesis reaction of 2-nitrostyrene. The two phenyl rings in 8 can be unsubstituted or substituted with electron withdrawing or donating group, including, but not limited to, halogen or alkoxyl respectively.

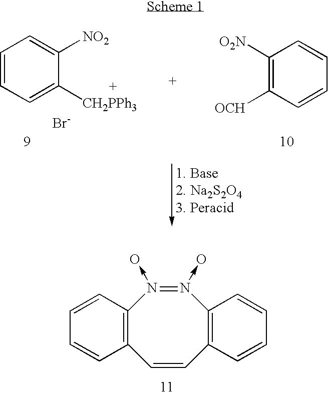

In another embodiment, compounds of Formula 8 wherein E is a single bond may be prepared in a similar manner from 9 and 10 as outlined in Scheme 2.

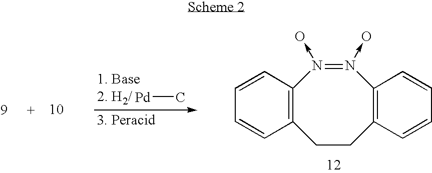

In another embodiment, compounds of Formula 8 wherein E is an aromatic or heteroaromatic moiety may be prepared according to Scheme 3.

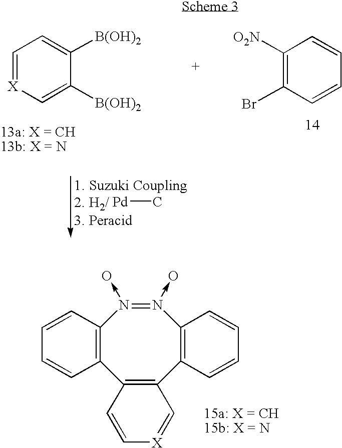

The coupling reaction between 13 a, b and 14 to generate 15 is carried out under Suzuki conditions where boronic acid derivative is treated with the halide in the presence of a base such as triphenylphosphine, potassium carbonate, sodium acetate, or triethyl amine, and in the presence of transition metal catalyst such as palladium acetate. The solvent for the reaction may be an aromatic hydrocarbon such as benzene or toluene; an ether such as tetrahydrofuran or dioxane; a halogenated hydrocarbon such as methylene chloride, chloroform, 1,2-dichloroethane; an aprotic polar solvent such as acetonitrile or dimethylformamide or a protic polar solvent such as ethanol or methanol. The reaction temperature is usually from 0° to +150° C., preferably from 50° to 120° C. The reaction time is from 1 to 24 hours.

Other 5-membered ring heteroaromatic compounds can be prepared in a similar manner (Scheme 4) by the Suzuki coupling of 14 with bis-boronic acid derivatives (14c and 14d) of 2,3- or 3,4-disubstituted furan, pyrrole, pyrazole, imidazole, thiazole, or oxazole followed by the reduction of the nitro group and oxidation to the diazocinedioxide.

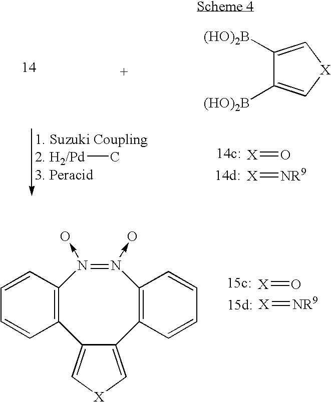

A general method of preparing various alicylic derivatives of Formula 8 is through the Diels-Alder reaction shown in Scheme 5. Both carbocylic and heterocyclic rings can be generated depending on the type of dienes used in the reaction. For example, if 1,3-butadiene or substituted 1,3-butadienes are used, then the Diels-Alder product will contain a carbocyclic ring. If azabutadiene or substituted azabutadienes are used, the reaction product will contain a heterocyclic ring.

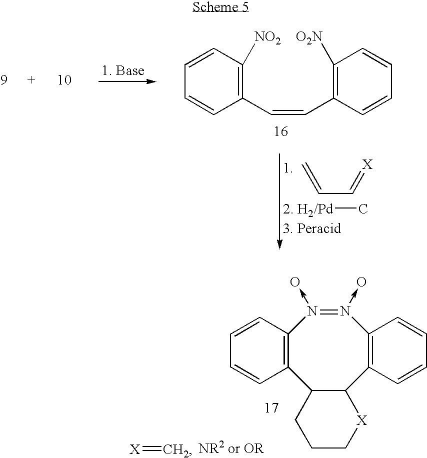

Three-membered carbocyclic ring can also be introduced by reacting 16 with trimethylsulfoxonium iodide/potassium t-butoxide in dimethylsulfoxide (DMSO) (Scheme 6). An aziridine ring can be introduced by thermal or photochemical insertion of nitrene into the double bond in 16. An epoxide ring can also be introduced by reacting 16 with peracids. The double bond in 16 can be transformed into other functional groups by the methods well known in the art.

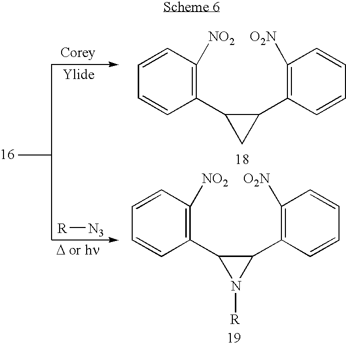

The bis thiophenol type derivative such as 20 can be prepared from 16 via the formation of a diazonium salt followed by treatment with sodium thiolate according to the procedures described in literature (Scheme 7). Conversion of 20 to the cyclic disulfide can be carried out under mild oxidative conditions such as treatment with iron chloride under acidic conditions or with hydrogen peroxide, and further oxidation of the disulfide to the dioxide 21 can be effected with hydrogen peroxide or peracids. The reaction is carried out in polar, aprotic or protic solvents such as methyl ethyl ketone, dioxane, ethanol or methanol at a temperature from 0° to +150° C., preferably from 0° to 50° C. The reaction time is from 1 to 4 hours.

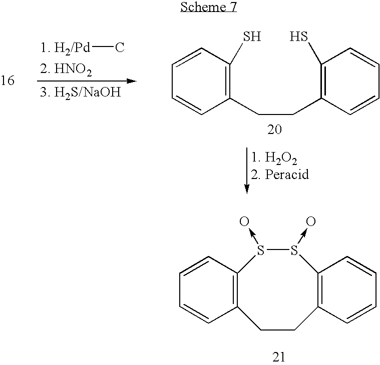

D. Pharmaceutical Compositions

The compounds provided herein can be used as such, be administered in the form of pharmaceutically acceptable salts derived from inorganic or organic acids, or used in combination with one or more pharmaceutically acceptable excipients. The phrase “pharmaceutically acceptable salt” means those salts which are, within the scope of sound medical judgment, suitable for use in contact with the tissues without undue toxicity, irritation, allergic response, and the like, and are commensurate with a reasonable benefit/risk ratio. Pharmaceutically acceptable salts are well known in the art. The salts can be prepared either in situ during the final isolation and purification of the compounds provided herein or separately by reacting the acidic or basic drug substance with a suitable base or acid respectively. Typical salts derived from organic or inorganic acids salts include, but are not limited to hydrochloride, hydrobromide, hydroiodide, acetate, adipate, alginate, citrate, aspartate, benzoate, bisulfate, gluconate, fumarate, hydroiodide, lactate, maleate, oxalate, palmitoate, pectinate, succinate, tartrate, phosphate, glutamate, and bicarbonate. Typical salts derived from organic or inorganic bases include, but are not limited to lithium, sodium, potassium, calcium, magnesium, ammonium, monoalkylammonium such as meglumine, dialkylammonium, trialkylammonium, and tetralkylammonium.

The mode of administration of the pharmaceutical compositions can be oral, rectal, intravenous, intramuscular, intracisternal, intravaginal, intraperitoneal, bucal, subcutaneous, intrasternal, nasal, or topical. The compositions can also be delivered at the target site through a catheter, an intracoronary stent (a tubular device composed of a fine wire mesh), a biodegradable polymer, or biological carriers including, but are not limited to antibodies, biotin-avidin complexes, and the like. Dosage forms for topical administration of a compound provided herein include powders, sprays, ointments and inhalants. The active compound is mixed under sterile conditions with a pharmaceutically acceptable carrier and any needed preservatives, buffers or propellants. Opthalmic formulations, eye ointments, powders and solutions are also provided herein.

Actual dosage levels of active ingredients and the mode of administration of the pharmaceutical compositions provided herein can be varied in order to achieve the effective therapeutic response for a particular patient. The phrase “therapeutically effective amount” of the compound provided herein means a sufficient amount of the compound to treat disorders, at a reasonable benefit/risk ratio applicable to any medical treatment. It will be understood, however, that the total daily usage of the compounds and compositions of the provided will be decided by the attending physician within the scope of sound medical judgment. The total daily dose of the compounds provided herein may range from about 0.0001 to about 1000 mg/kg/day. For purposes of oral administration, doses can be in the range from about 0.001 to about 5 mg/kg/day. If desired, the effective daily dose can be divided into multiple doses for purposes of administration; consequently, single dose compositions may contain such amounts or submultiples thereof to make up the daily dose. The specific therapeutically effective dose level for any particular patient will depend upon a variety of factors including the disorder being treated and the severity of the disorder; medical history of the patient, activity of the specific compound employed; the specific composition employed, age, body weight, general health, sex and diet of the patient, the time of administration, route of administration, the duration of the treatment, rate of excretion of the specific compound employed, drugs used in combination or coincidental with the specific compound employed; and the like.

The compounds provided can be formulated together with one or more non-toxic pharmaceutically acceptable diluents, carriers, adjuvants, and antibacterial and antifungal agents such as parabens, chlorobutanol, phenol, sorbic acid, and the like. Proper fluidity can be maintained, for example, by the use of coating materials such as lecithin, by the maintenance of the required particle size in the case of dispersions, and by the use of surfactants. In some cases, in order to prolong the effect of the drug, it is desirable to decrease the rate of absorption of the drug from subcutaneous or intramuscular injection. This can be accomplished by suspending crystalline or amorphous drug substance in a vehicle having poor water solubility such as oils. The rate of absorption of the drug then depends upon its rate of dissolution, which, in turn, may depend upon crystal size and crystalline form. Prolonged absorption of an injectable pharmaceutical form can be achieved by the use of absorption delaying agents such as aluminum monostearate or gelatin.

The compound provided herein can be administered enterally or parenterally in solid or liquid forms. Compositions suitable for parenteral injection may comprise physiologically acceptable, isotonic sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, or mixtures of these substances.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or (a) fillers or extenders such as starches, lactose, sucrose, glucose, mannitol and silicic acid; (b) binders such as carboxymethylcellulose, alginates, gelatin, polyvinylpyrrolidone, sucrose and acacia; (c) humectants such as glycerol; (d) disintegrating agents such as agar-agar, calcium carbonate, potato or tapioca starch, alginic acid, certain silicates and sodium carbonate; (e) solution retarding agents such as paraffin; (f) absorption accelerators such as quaternary ammonium compounds; (g) wetting agents such as cetyl alcohol and glycerol monostearate; (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds provided herein with suitable non-irritating excipients or carriers such as cocoa butter, polyethylene glycol or a suppository wax which are solid at room temperature but liquid at body temperature and therefore melt in the rectum or vaginal cavity and release the active compound.

Compounds provided herein can also be administered in the form of liposomes. Methods to form liposomes are known in the art (Prescott, Ed., Methods in Cell Biology 1976, Volume XIV, Academic Press, New York, N.Y.) As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound provided herein, stabilizers, preservatives, excipients and the like. The preferred lipids are natural and synthetic phospholipids and phosphatidyl cholines (lecithins).

The compounds provided herein can also be administered in the form of a ‘prodrug’ wherein the active pharmaceutical ingredients, represented by Formulas 1-3, are released in vivo upon contact with hydrolytic enzymes such as esterases and phophatases in the body. The term “pharmaceutically acceptable prodrugs” as used herein represents those prodrugs of the compounds provided herein, which are, within the scope of sound medical judgment, suitable for use in contact with the tissues without undue toxicity, irritation, allergic response, and the like, commensurate with a reasonable benefit/risk ratio, and effective for their intended use. A thorough discussion is provided in T. Higuchi and V. Stella (Higuchi, T. and Stella, V. Pro-drugs as Novel Delivery Systems, V. 14 of the A.C.S. Symposium Series; Edward B. Roche, Ed., Bioreversible Carriers in Drug Design 1987, American Pharmaceutical Association and Pergamon Press), which is incorporated herein by reference.

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or pharmaceutically acceptable derivatives may be packaged as articles of manufacture containing packaging material, a compound or pharmaceutically acceptable derivative thereof provided herein, which is effective for modulating the activity of a Bcl-2 protein, or for treatment, prevention or amelioration of one or more symptoms of Bcl-2 protein-mediated diseases or disorders, or diseases or disorders in which Bcl-2 protein-mediated activity, is implicated, within the packaging material, and a label that indicates that the compound or composition, or pharmaceutically acceptable derivative thereof, is used for modulating the activity of a Bcl-2 protein, or for treatment, prevention or amelioration of one or more symptoms of Bcl-2 protein-mediated diseases or disorders, or diseases or disorders in which Bcl-2 protein activity is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which a Bcl-2 protein is implicated as a mediator or contributor to the symptoms or cause.

E. Evaluation of the Activity of the Compounds

The biological activity of the compounds provided herein were assessed in cellular system by the method similar to the one described by Amundson et al. (Amundson, S. A. et al, Cancer Research 2000, 60, 6101-6110), which is incorporated herein by reference in its entirety. The compounds were tested for acute cytotoxicity (apoptotic activity) using two human cancer cell lines expressing high levels of either Bcl-2 or Bcl-X_{L}. HL60, human leukemic cells expressing high Bcl-2 and low Bcl-X_{L} levels, and A549 human lung cells expressing low Bcl-2 and high Bcl-X_{L} levels, were incubated with the compounds at concentrations between 0.5 and 1000 μg/mL for 4 hours at 37° C., 5% CO₂. The number of viable cells were measured by mitochondrial transformation of Alamar Blue to a fluorescent dye. LC₅₀ values for each test compound and controls are provided in the individual examples that follow.

F. Combination Therapy

The compounds provided herein may be administered as the sole active ingredient or in combination with other active ingredients. Other active ingredients that may be used in combination with the compounds provided herein include known Bcl-2 protein antagonists, other compounds for use in treating, preventing, or ameliorating one or more symptoms of Bcl-2 protein mediated diseases and disorders, anti-angiogenesis agents, anti-tumor agents, and other cancer treatments. Such compounds include, in general, but are not limited to, alkylating agents, toxins, antiproliferative agents and tubulin binding agents. Classes of cytotoxic agents for use herein include, for example, the anthracycline family of drugs, the vinca drugs, the mitomycins, the bleomycins, the cytotoxic nucleosides, the pteridine family of drugs, diynenes, the maytansinoids, the epothilones, the taxanes and the podophyllotoxins.

G. Methods of Use of the Compounds and Compositions

The compounds and compositions provided herein are useful in methods of treatment, prevention, or amelioration of one or more symptoms of Bcl-2 protein-mediated diseases or disorders, including but not limited to Bcl-2 and Bcl-X_{L} mediated diseases or disorders. In certain embodiments, the diseases are characterized by over-expression of to Bcl-2 and Bcl-XL protein. In certain embodiments the diseases or disorders include, but are not limited to, cancers, tumors, hyperproliferative diseases, acquired immune deficiency syndrome, degenerative conditions, and vascular diseases. In certain embodiments, the cancers include, but are not limited to B-cell lymphoma including B-cell lymphoma-2, B-cell leukemia, skin cancer, pancreatic cancer, ovarian cancer, liver cancer, bladder cancer, adrenal carcinoma, breast cancer, prostate cancer, colorectal cancer including colorectal adenocarcinomas, follicular lymphoma.

Methods of modulating the activity of a Bcl-2 protein, including but not limited to an anti-apoptotic Bcl-2 protein, Bcl-2 and Bcl-X_{L}, by administering one or more of the compounds or compositions provided herein are also provided.

Methods of antagonizing a Bcl-2 protein, including but not limited to an anti-apoptotic Bcl-2 protein, including Bcl-2 and Bcl-X_{L}, by contacting a composition containing the Bcl-2 protein with one or more of the compounds or compositions provided herein are also provided.

Methods of altering the interaction of an anti-apoptotic Bcl-2 protein, including but not limited to Bcl-2 and Bcl-X_{L}, and a pro-apoptotic Bcl-2 protein, including but not limited to Bax, Bak, Bid and Bad, by contacting a composition containing the anti-apoptotic Bcl-2 protein and the pro-apoptotic Bcl-2 protein with a compound or composition provided herein, are also provided.

Methods of inducing apoptosis by administering one or more of the compounds or compositions provided herein are also provided.

The following examples are exemplary only and are not intended to limit the scope of the subject matter claimed herein.

### EXAMPLE 1

Preparation of dibenzo[c,g][1,2]diazocine, 11,12-dihydro-2,9-bis(2,2,2-trifluoroethoxy)-, 5,6-dioxide and dibenzo[c,g][1,2]diazocine, 11,12-dihydro-2,9-bis(2,2,2-trifluoroethoxy)-, 5-oxide

Step 1. A mixture of 3-methyl-4-nitrophenol (2.5 g, 16.3 mmol), 2,2,2-trifluoroiodoehtnae (4.1 g, 19.6 mmol), potassium carbonate (2.7 g, 19.6 mmol) in anhydrous dimethylformamide (50 ml) was stirred at 110° C. for 2 hr. Additional 2,2,2-trifluoroiodoethnae (1.0 g, 4.8 mmol) was added and solution stirred at 110° C. for 1 hr. Solution was added to water and product extracted with ethyl acetate. Chromatography on silica gel (hexane:ethyl acetate, 9:1) afforded 2-methyl-1-nitro-4-(2,2,2-trifluoro-ethoxy)-benzene. (2.9 g, 12.33 mmol). ¹H NMR (CDCl₃) 2.67 (3H, s), 4.45 (2H, q, J=7.8 Hz), 6.83-6.86 (2H, m), 8.12 (1H, d, J=9.6 Hz) ppm.

Step 2. Potassium t-butoxide (0.59 g, 5.24 mmol) was suspended in anhydrous ether (10 ml) and dimethylsulfoxide (0.5 ml) and stirred at −20° C. Compound 2 (1.07 g, 4.56 mmol) was added in portions and solution stirred at −10° C. for 2 hr. Water was added and the product extracted with ethyl acetate. Flash chromatography on silica gel (hexane:ethyl acetate, 90:10-85:15) afforded 4-(2,2,2-trifluoroethoxy)-2-[2-[5-(2,2,2-trifluoroethoxy)-2-nitrophenyl]ethyl]-1-nitrobenzene (0.63 g, 1.35 mmol). LC-MS (API-ES, pos.) M+Na⁺ 491.0; ¹H NMR (CDCl₃) 3.31 (4H, s), 4.47 (4H, q, J=7.8 Hz), 6.92-7.0 (4H, m), 8.14 (2H, d, J-9.3 Hz) ppm.

Step 3. Hydrazine hydrate (0.25 g, 7.8 mmol) was added to a mixture of 3 (0.51 g, 1.09 mmol) and 10% Pd on carbon (0.05 g) in 95% ethanol (25 ml). After refluxing for 3 hr, the catalyst was filtered and washed with methanol. Combined filtrate was evaporated and residue was crystallized from methylene chloride-hexane to afford 1-amino-4-(2,2,2-trifluoroethoxy)-2-[2-[2-amino-5-(2,2,2-trifluoroethoxy)phenyl]ethyl]benzene (0.37 g, 0.92 mmol). LC-MS (API-ES, pos.) M+H⁺ 409.1; ¹H NMR (DMSO-d6) 2.64 (4H, s), 4.53 (4H, q, J=9.0 Hz), 4.63 (4H, br s), 6.60 (2H, d, J=8.7 Hz), 6.66 (2H, dd, J=2.7, 8.7 Hz), 6.79 (2H, d, 2.7 Hz) ppm.

Step 4. To a stirred suspension of 4 (0.33 g, 0.81 mmol) and sodium tungstate dihydrate (0.037 g, 0.11 mmol) in 95% ethanol (3 ml) and water (1 ml), 30% hydrogen peroxide (0.46 g, 4.05 mmol) was added under ice cooling. The solution was stirred at this temperature for 4 hr followed by stirring at ambient temperature over night. TLC analysis indicated complete disappearance of the starting material. The mixture was adsorbed on silica gel and subjected to chromatography. Elution with hexane-ethyl acetate (75:25) afforded dibenzo[c,g][1,2]diazocine, 11,12-dihydro-2,9-bis(2,2,2-trifluoroethoxy)-, 5-oxide (0.06 g, 0.14 mmol). LC-MS (API-ES, pos.) M+H⁺ 421.0, M+Na⁺ 443.0; ¹H NMR (CDCl₃) 2.78-3.0 (2H, m), 3.18-3.44 (2H, m), 4.28 (2H, q, J=8.4 Hz), 4.30 (2H, q, J=7.8 Hz), 6.63 (1H, d, J=2.7 Hz), 6.66 (1H, d, J=2.7 Hz), 6.70-6.76 (2H, m), 6.91 (1H, d, J=8.4 Hz), 7.17 (1H, d, J=8.7 Hz) ppm. Elution with hexane-ethyl acetate (60:40) furnished light green colored fractions containing dibenzo[c,g][1,2]diazocine, 11,12-dihydro-2,9-bis(2,2,2-trifluoroethoxy)-, 5,6-dioxide which was crystallized from 95% ethanol (0.13 g, 0.31 mmol). ¹H NMR in deuterated chloroform indicated it to be present as an equilibrium mixture of ring cyclized isomer (diazocine dioxide form) and ring open isomer (bisnitroso form) in a ratio of approximately 1:1.5. LC-MS (API-ES, pos.) M+Na⁺ 459.1; ¹H NMR (CDCl₃) (diazocine dioxide from) 3.01 (2H, m), 3.36 (2H, m), 4.33 (4H, q, J=8.1 Hz), 6.70 (2H, d, J=2.7 Hz), 6.81 (2H, dd, J=2.7, 9.0 Hz), 7.39 (2H, d, J=9.0 Hz) ppm. ¹H NMR (CDCl₃) (bisnitroso from) 4.41 (4H, s), 4.43 (4H, q, J=8.1 Hz), 6.42 (2H, d, J=9.0 Hz), 6.74 (2H, dd, J=2.7, 9.0 Hz), 6.99 (2H, d, J=2.7 Hz) ppm.

The LC₅₀ value for this compound in A549 and HL-60 cell lines were 80.91 μM and 26.13 μM respectively.

### EXAMPLE 2

Preparation of dibenzo[c,g][1,2]diazocine, 2,9-bis(difluoromethoxy)-11,12-dihydro-5,6-dioxide

Step 1. A mixture of 3-methyl-4-nitrophenol (1.0 g, 6.53 mmol) and sodium hydroxide (0.39 g, 9.8 mmol) in dioxane (50 ml) and water (5 ml) was stirred at 70° C. Chlorodifluoromethane gas was bubbled through the solution with stirring (equipped with a dry ice condensor) for 0.5 hr. Water was added and product extracted with ethyl acetate. Removal of the solvent afforded a residue which was subjected to column chromatography on silica gel. Elution with hexane:ethyl acetate (9:1) afforded 4-(difluoromethoxy)-2-methyl-1-nitrobenzene (0.9 g, 4.43 mmol). ¹H NMR (CDCl₃) 2.66 (3H, s), 6.63 (1H, t, J=72.3 Hz), 7.08-7.12 (2H, m), 8.09 (1H, d, J=9.6 Hz) ppm.

Step 2. Potassium t-butoxide (1.59 g, 14.17 mmol) was suspended in anhydrous ether (13 ml) and dimethylsulfoxide (0.7 ml) and stirred at −10° C. 4-(Difluoromethoxy)-2-methyl-1-nitrobenzene (2.5 g, 12.31 mmol) was added in portions and solution stirred at −10° C. for 1 hr followed by stirring at ambient temperature for 2 hr. Water was added and the product extracted with ethyl acetate. Flash chromatography on silica gel (hexane:ethyl acetate, 8:2) afforded 4-(difluoromethoxy)-2-[2-[5-(difluoromethoxy)-2-nitrophenyl]ethyl]-1-nitrobenzene (0.54 g, 1.34 mmol). LC-MS (API-ES, pos.) M+Na⁺ 427.0; ¹H NMR (CDCl₃) 3.32 (4H, s), 6.62 (2H, t, J=72.3 Hz), 7.08-7.18 (4H, m), 8.10 (2H, d, J=8.7 Hz) ppm.

Step 3. Hydrazine hydrate (0.29 g, 9.17 mmol) was added to a mixture of 4-(difluoromethoxy)-2-[2-[5-(difluoromethoxy)-2-nitrophenyl]ethyl]-1-nitrobenzene (0.53 g, 1.31 mmol) and 10% Pd on carbon (0.05 g) in 95% ethanol (25 ml). After refluxing for 3 hr, the catalyst was filtered and washed with methanol. Combined filtrate was evaporated and residue was crystallized from ethanol to afford 1-amino-4-(difluoromethoxy)-2-[2-[2-amino-5-(difluoromethoxy)phenyl]ethyl]benzene (0.21 g, 0.61 mmol). LC-MS (API-ES, pos.) M+H⁺ 345.1; ¹H NMR (CDCl₃) 2.80 (4H, s), 3.61 (4H, br s), 6.36 (2H, t, J=75.0 Hz), 6.66 (2H, d, J=8.4 Hz), 6.82 (2H, d, 2.7 Hz), 6.87 (2H, dd, J=2.7, 8.4 Hz) ppm.

Step 4. To a stirred suspension of 1-amino-4-(difluoromethoxy)-2-[2-[2-amino-5-(difluoromethoxy)phenyl]ethyl]benzene (0.45 g, 1.31 mmol) and sodium tungstate dihydrate (0.06 g, 0.18 mmol) in 95% ethanol (3 ml) and water (1 ml), 30% hydrogen peroxide (0.93 g, 8.2 mmol) was added under ice cooling. The solution was stirred at this temperature for 1 hr followed by stirring at ambient temperature over night. Solvent was removed and the residue chromatographed on silica gel in methylene chloride:methanol (98:2). The fraction containing the desired material was further purified by HPLC (column, Alltech Econosil C18, 10 μm, 10×250 mm, acetonitrile:water, 1:1 to acetonitrile, 15 min, 2.5 ml/min) followed by crystallization form ether-hexane to afford dibenzo[c,g][1,2]diazocine, 2,9-bis(difluoromethoxy)-11,12-dihydro-, 5,6-dioxide (0.07 g, 0.19 mmol). ¹H NMR in deuterated chloroform indicated it to be present as an equilibrium mixture of ring cyclized isomer (diazocine dioxide form) and ring open isomer (bisnitroso form) in a ratio of approximately 2.9:1.0. LC-MS (API-ES, pos.) M+Na⁺ 395.0; ¹H NMR (CDCl₃) (diazocine dioxide from) 3.06 (2H, m), 3.36 (2H, m), 6.51 (2H, t, J=72.6 Hz), 6.90 (2H, d, J=2.4 Hz), 7.03 (2H, dd, J=2.4, 8.4 Hz), 7.45 (2H, d, J=8.4 Hz) ppm. ¹H NMR (CDCl₃) (bisnitroso from) 4.46 (4H, s), 6.28 (2H, d, J=8.4 Hz), 6.59 (2H, t, J=72.6 Hz), 6.88 (2H, dd, J=2.1, 8.4 Hz), 7.16 (2H, d, J=2.1 Hz) ppm.

The LC₅₀ value for this compound in A549 and HL-60 cell lines were 298.44 μM and 18.53 μM respectively.

### EXAMPLE 3

The compounds provided herein were tested for acute cytoxicity (apoptotic activity) using two human cancer cell lines expressing high levels of either Bcl-2 or Bcl-X_{L}. HL-60 cells expressing high Bcl-2 and low Bcl-X_{L} levels, and A549 human lung cells expressing low Bcl-2 and high Bcl-X_{L} levels (Amundson, S. A. et al. Cancer Research 2000, 60, 6101-6110) were incubated with the compounds at concentrations between 0.5 and 1000 mg/mL for 4 hours at 37° C., 5% CO₂. The number of viable cells were measured my mitochondrial transformation of Alamar Blue to a fluorescent dye. As negative controls, the compounds were tested against the cell lines with low levels of Bcl-2 expression, viz., MDA-453 cells (Enyedy, I. J. et al. J. Med. Chem. 2001, 44, 4313-4324). LC₅₀ values for each compound and controls were determined.

Since modifications will be apparent to those of skill in the art, it is intended that the subject matter claimed herein be limited only by the scope of the appended claims.

We claim:

1. A compound of Formula 8, 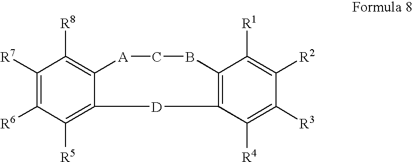
or a pharmaceutically acceptable derivative thereof, wherein:
A and B are each independently selected from the group consisting of —N—, —NO—, —SO—, —SO₂—, and —NR⁹—;
C is a single bond or a double bond;
D is selected from the group consisting of
single bond, 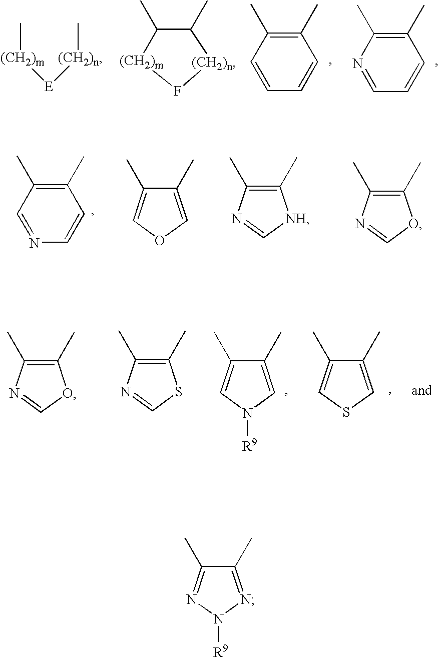
E and F are each independently selected from the group consisting of single bond, double bond, —NR⁹—, —CR¹⁰R¹¹—, —O—, —S—, —SO—, and —SO₂—;
m and n are each independently an integer from 0 to 6;
R¹ to R⁸, R¹⁰, and R¹¹ are each independently selected from the group consisting of hydrogen, C1-C10 alkyl, C3-C10 cycloalkyl, C2-C6 alkenyloxyl, C3-C8 cycloalkoxyl, C1-C10 acyl, C5-C10 aryl, C5-C10 aryloxy, C1-C10 alkoxy, C1-C10 alkoxyalkyl, C1-C10 aralkoxy, C1-C10 heteroaralkoxy, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, C1-C10 alkylcarbonylamino, C1-C10 mono- or polyhaloalkylcarbonylamino, halo, mono- or polyhaloalkyl, mono- or polyhaloalkoxyl, cyano, nitro, mercapto, C1-C10 mercaptoalkyl, C1-C10 thioalkyl, C1-C10 alkylthioalkyl, C1-C10 sulfonylalkyl, and C1-C10 alkylsulfonylalkyl; and
R⁹ is selected from the group consisting of hydrogen, C1-C10 alkyl, C3-C10 cycloalkyl, C1-C10 acyl, C5-C10 aryl, C1-C10 alkoxyalkyl, C1-C10 aminoalkyl, C1-C10 alkylaminoalkyl, hydroxyl, C1-C10 hydroxyalkyl, C1-C10 carboxyalkyl, C1-C10 alkoxylcarbonyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl; with the proviso that if E is a single bond, A and B are both —NO— and m and n are both 1, and (i) if R¹, R², R⁴, R⁵, R⁷ and R⁸ are H, then R³ and R⁶ both are not hydrogen, hydroxyl, halo, alkoxyl, alkenyloxyl, cycloalkoxyl, phenoxyl, or trifluoromethoxyl and (ii) if R¹, R³-R⁶ and R⁸ are H, then R² and R⁷ both are not methyl, halo, and methoxycarbonyl.

2. The compound of claim 1, or a pharmaceutically acceptable derivative thereof, wherein:
A and B are each independently —NO—, or —N—;
C is a double bond;
D is selected from the group consisting of:
single bond, 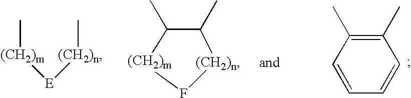
E and F are each independently selected from the group consisting of single bond, double bond, —NR⁹—, —CR¹⁰R¹¹—, and —O—;
m and n are each independently and integer from 0 to 6;
R¹ to R⁸, R¹⁰, and R¹¹ are each independently selected from the group consisting of hydrogen, C1-C10 alkyl, C5-C10 aryl, C1-C10 alkoxyl, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, halogen, mono- or polyfluroalkyl, mono- or polyfluroalkoxyl, cyano, nitro, C1-C10 thioalkyl, C1-C10 sulfonylalkyl, and C1-C10 alkylsulfonylalkyl; and
R⁹ is selected from the group consisting of hydrogen, C1-C10 alkyl, C3-C10 cycloalkyl, C1-C10 acyl, C5-C10 aryl, C1-C10 alkoxyalkyl, C1-C10 hydroxyalkyl, C1-C10 carboxyalkyl, C1-C10 alkoxylcarbonyl, and C1-C10 alkoxycarbonylalkyl, with the proviso that if E is a single bond, A and B are both —NO— and m and n are both 1, and (i) if R¹, R², R⁴, R⁵, R⁷ and R⁸ are H, then R³ and R⁶ both are not hydrogen, hydroxyl, halo, alkoxyl, phenoxyl, or trifluoromethoxyl and (ii) if R¹, R³-R⁶ and R⁸ are H, then R² and R⁷ both are not methyl, halo, and methoxycarbonyl.

3. The compound of claim 1, or a pharmaceutically acceptable derivative thereof, wherein:
A and B are —NO—;
C is a double bond;
D is selected from the group consisting of:
single bond, 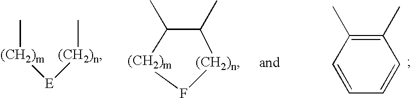
E and F are each independently single bond, double bond, —NR⁹—, or —O—;
m and n are each independently and integer from 0 to 6;
R¹ to R⁸, R¹⁰, and R¹¹ are each independently selected from the group consisting of hydrogen, C1-C10 alkyl, C5-C10 aryl, C1-C10 alkoxyl, amino, hydroxyl, C1-C10 hydroxyalkyl, carboxyl, C1-C10 alkoxylcarbonyl, mono- or polyfluroalkyl, mono- or polyfluroalkoxyl, cyano, nitro, C1-C10 thioalkyl, and C1-C10 sulfonylalkyl; and
R⁹ is selected from the group consisting of hydrogen, C1-C1 alkyl, C3-C10 cycloalkyl, C1-C10 acyl, C5-C10 aryl, carboxyalkyl, C1-C10 alkoxylcarbonyl, and C1-C10 alkoxycarbonylalkyl, with the proviso that if E is a single bond, A and B are both —NO— and m and n are both 1, and (i) if R¹, R², R¹, R⁵, R⁷ and R⁸ are H, then R³ and R⁶ both are not hydrogen, hydroxyl, alkoxyl or trifluoromethoxyl and (ii) if R¹, R³-R⁶ and R⁸ are H, then R² and R⁷ both are not methyl.

4. The compound of claim 1, or a pharmaceutically acceptable derivative thereof, wherein:
A and B are —NO—;
C is a double bond;
D is selected from the group consisting of:
single bond, 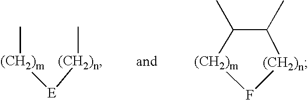
E and F are each independently single bond, double bond, —NR⁹—, or —O—;
m and n are each independently an integer from 0 to 6;
R¹ to R⁸, R¹⁰, and R¹¹ are each independently selected from the group consisting of hydrogen, C1-C10 alkyl, C1-C10 alkoxyl, mono- or polyfluroalkyl, and mono- or polyfluroalkoxyl; and
R⁹ is selected from the group consisting of hydrogen, C1-C10 alkyl, C3-C10 cycloalkyl, C1-C10 acyl, and C5-C10 aryl, and C1-C10 alkoxylcarbonyl, with the proviso that if E is a single bond, A and B are both —NO— and m and n are both 1, and (i) if R¹, R², R⁴, R⁵, R⁷ and R⁸ are H, then R³ and R⁶ both are hydrogen, alkoxyl, or trifluoromethoxyl and (ii) if R¹, R³-R⁶ and R⁸ are H, then R² and R⁷ both are not methyl.

5. The compound of claim 1, or a pharmaceutically acceptable derivative thereof, wherein A and B are —SO—; C is a single bond;
D is selected from the group consisting of:
single bond, 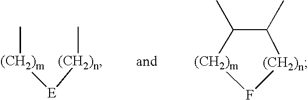
E and F are each independently single bond, double bond, —NR⁹—, or —O—;
m and n are each independently an integer from 0 to 6;
R¹ to R⁸, R¹⁰, and R¹¹ are each independently selected from the group consisting of hydrogen, C1-C10 alkyl, C1-C10 alkoxyl, mono- or polyfluroalkyl, and mono- or polyfluroalkoxyl; and
R⁹ is selected from the group consisting of hydrogen, C1-C10 alkyl, C3-C10 cycloalkyl, C1-C10 acyl, and C5-C10 aryl, and C1-C10 alkoxylcarbonyl.

6. The compound of claim 1, or a pharmaceutically acceptable derivative thereof, wherein at least one of R¹-R⁸ is electron withdrawing substituents.

7. The compound of claim 1, or a pharmaceutically acceptable derivative thereof, wherein at least two of R¹-R⁸ are electron withdrawing substituents.

8. The compound of claim 1, or a pharmaceutically acceptable derivative thereof, wherein at least one of R¹-R⁸ is selected from haloalkoxy, alkylsulfonyl and haloamido groups.

9. The compound of claim 1, or a pharmaceutically acceptable derivative thereof, wherein at least two of R¹-R⁸ are selected from haloalkoxy, alkylsulfonyl and haloamido groups.

10. The compound of claim 1, or a pharmaceutically acceptable derivative thereof, wherein R¹, R⁴, R⁵ and R⁸ are each hydrogen.

11. The compound of claim 1, or a pharmaceutically acceptable derivative thereof, wherein, -A-C—B— is —NO═NO—.

12. The compound of claim 1, wherein D is alkylene or a single bond.

13. The compound of claim 1, wherein D is ethylene or a single bond.

14. The compound of claim 1, wherein R² and R⁷ are each independently hydrogen or C1-C10 polyhaloalkylcarbonylamino.

15. The compound of claim 1, wherein R² and R⁷ are each independently hydrogen or trifluoromethylcarbonylamino.

16. The compound of claim 1, wherein R² and R⁷ are each hydrogen.

17. The compound of claim 1, wherein R² and R⁷ are each trifluoromethylcarbonylamino.

18. The compound of claim 1, wherein R³ and R⁶ are each independently hydrogen, polyhaloalkoxy, halo, aralkoxy, alkylsulfonyl or polyhaloalkylheteroaryloxy.

19. The compound of claim 1, wherein R³ and R⁶ are each independently hydrogen, difluoromethoxy, 2,2,2-trifluoroethoxy, 1,1,2-trifluoro-2-chloroethoxy, 1,1,2,3,3,3-hexafluoropropoxy, fluoro, benzyloxy, ethanesulfonyl or 5-trifluoromethyl-2-pyridyloxy.

20. The compound of claim 1, wherein R³ and R⁶ are each independently polyhaloalkoxy, halo, aralkoxy, alkylsulfonyl or polyhaloalkylheteroaryloxy.

21. The compound of claim 1, wherein R³ and R⁶ are each independently difluoromethoxy, 2,2,2-trifluoroethoxy, 1,1,2-trifluoro-2-chloroethoxy, 1,1,2,3,3,3-hexafluoropropoxy, fluoro, benzyloxy, ethanesulfonyl or 5-trifluoromethyl-2-pyridyloxy.

22. The compound of claim 1, wherein the compound has formula: 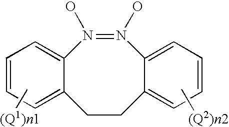
wherein n₁ and n₂ are each independently an integer from 1 to 4 and Q¹ and Q² are each independently selected from haloalkoxy, alkylsulfonyl and haloamido groups, with the proviso that Q¹ and Q² are both not trifluoromethoxyl.

23. The compound of claim 22, wherein the compound has formula:

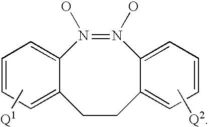

24. An article of manufacture, comprising packaging material, a compound of formula 8: 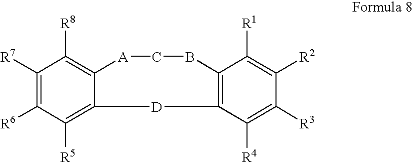
or a pharmaceutically acceptable derivative thereof, wherein:
A and B are each independently selected from the group consisting of —N—, —NO—, —SO—, —SO₂—, and —NR⁹—;
C is a single bond or a double bond;
D is selected from the group consisting of
single bond, 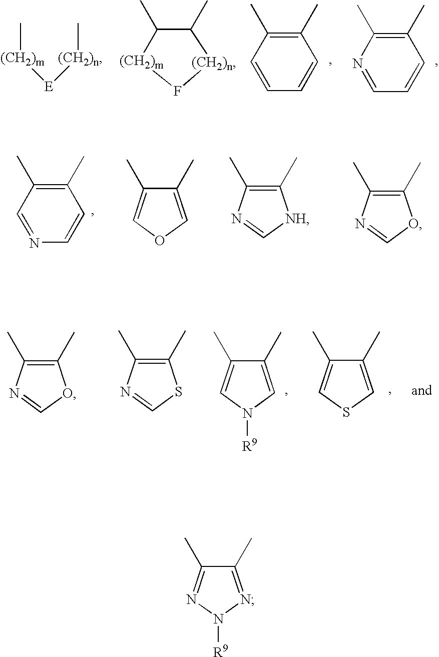
E and F are each independently selected from the group consisting of single bond, double bond, —NR⁹—, —CR¹⁰R¹¹—, —O—, —S—, —SO—, and —SO₂—;
m and n are each independently an integer from 0 to 6;
R¹ to R⁸, R¹⁰, and R¹¹ are each independently selected from the group consisting of hydrogen, C1-C10 alkyl, C3-C10 cycloalkyl, C2-C6 alkenyloxyl, C3-C8 cycloalkoxyl, C₁-C10 acyl, C5-C10 aryl, C5-C10 aryloxy, C1-C10 alkoxy, C1-C10 alkoxyalkyl, C1-C10 aralkoxy, C1-C10 heteroaralkoxy, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, C1-C10 alkylcarbonylamino, C1-C10 mono- or polyhaloalkylcarbonylamino, halo, mono- or polyhaloalkyl, mono- or polyhaloalkoxyl, cyano, nitro, mercapto, C1-C10 mercaptoalkyl, C1-C10 thioalkyl, C1-C10 alkylthioalkyl, C1-C10 sulfonylalkyl, and C1-C10 alkylsulfonylalkyl; and
R⁹ is selected from the group consisting of hydrogen, C1-C10 alkyl, C3-C10 cycloalkyl, C1-C10 acyl, C5-C10 aryl, C1-C10 alkoxyalkyl, C1-C10 aminoalkyl, C1-C10 alkylaminoalkyl, hydroxyl, C1-C10 hydroxyalkyl, C1-C10 carboxyalkyl, C1-C10 alkoxylcarbonyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl; with the proviso that if E is a single bond, A and B are both —NO— and m and n are both 1, and (i) if R¹, R², R⁴, R⁵, R⁷ and R⁸ are H, then R³ and R⁶ both are not hydrogen, hydroxyl, halo, alkoxyl, alkenyloxyl, cycloalkoxyl, phenoxyl, or trifluoromethoxyl and (ii) if R¹, R³-R⁶ and R⁸ are H, then R² and R⁷ both are not methyl, halo, and methoxycarbonyl.

25. A pharmaceutical composition, comprising, in a pharmaceutically acceptable carrier, a compound of formula 8: 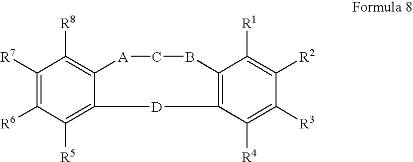
or a pharmaceutically acceptable derivative thereof, wherein:
A and B are each independently selected from the group consisting of —N—, —NO—, —SO—, —SO₂—, and —NR⁹—;
C is a single bond or a double bond;
D is selected from the group consisting of
single bond, 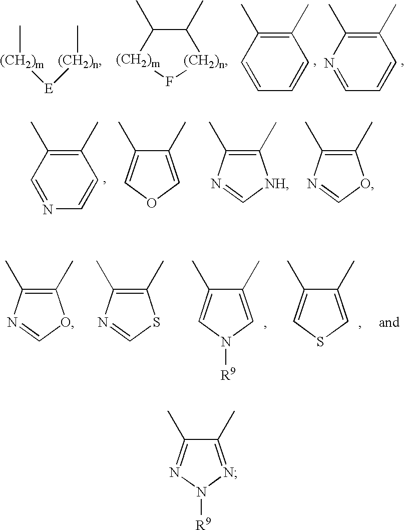
E and F are each independently selected from the group consisting of single bond, double bond, —NR⁹—, —CR¹⁰R¹¹—, —O—, —S—, —SO—, and —SO₂—;
m and n are each independently an integer from 0 to 6;
R¹ to R⁸, R¹⁰, and R¹¹ are each independently selected from the group consisting of hydrogen, C1-C10 alkyl, C3-C10 cycloalkyl, C2-C6 alkenyloxyl, C3-C8 cycloalkoxyl, C1-C10 acyl, C5-C10 aryl, C5-C10 aryloxy, C1-C10 alkoxy, C1-C10 alkoxyalkyl, C1-C10 aralkoxy, C1-C10 heteroaralkoxy, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, C1-C10 alkylcarbonylamino, C1-C10 mono- or polyhaloalkylcarbonylamino, halo, mono- or polyhaloalkyl, mono- or polyhaloalkoxyl, cyano, nitro, mercapto, C1-C10 mercaptoalkyl, C1-C10 thioalkyl, C1-C10 alkylthioalkyl, C1-C10 sulfonylalkyl, and C1-C10 alkylsulfonylalkyl; and
R⁹ is selected from the group consisting of hydrogen, C1-C10 alkyl, C3-C10 cycloalkyl, C1-C10 acyl, C5-C10 aryl, C1-C10 alkoxyalkyl, C1-C10 aminoalkyl, C1-C10 alkylaminoalkyl, hydroxyl, C1-C10 hydroxyalkyl, C1-C10 carboxyalkyl, C1-C10 alkoxylcarbonyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl; with the proviso that if E is a single bond, A and B are both —NO— and m and n are both 1, and (i) if R¹, R², R⁴, R⁵, R⁷ and R⁸ are H, then R³ and R⁶ both are not hydrogen, hydroxyl, halo, alkoxyl, alkenyloxyl, cycloalkoxyl, phenoxyl, or trifluoromethoxyl and (ii) if R¹, R³-R⁶ and R⁸ are H, then R² and R⁷ both are not methyl, halo, and methoxycarbonyl.

26. The composition of claim 25, wherein:
A and B are each independently —NO—, or —N—;
C is a double bond;
D is selected from the group consisting of:
single bond, 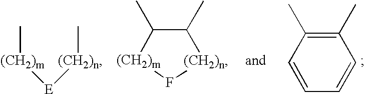
E and F are each independently selected from the group consisting of single bond, double bond, —NR⁹—, —CR¹⁰R¹¹—, and —O—;
m and n are each independently and integer from 0 to 6;
R¹ to R⁸, R¹⁰, and R¹¹ are each independently selected from the group consisting of hydrogen, C1-C10 alkyl, C5-C10 aryl, C1-C10 alkoxyl, amino, C1-C10 aminoalkyl, C1-C10 alkylaminoalkyl, hydroxyl, C1-C10 hydroxyalkyl, carboxyl, C1-C10 carboxyalkyl, C1-C10 alkoxylcarbonyl, C1-C10 alkoxycarbonylalkyl, halogen, mono- or polyfluroalkyl, mono- or polyfluroalkoxyl, cyano, nitro, C1-C10 thioalkyl, C1-C10 sulfonylalkyl, and C1-C10 alkylsulfonylalkyl; and
R⁹ is selected from the group consisting of hydrogen, C1-C10 alkyl, C3-C10 cycloalkyl, C1-C10 acyl, C5-C10 aryl, C1-C10 alkoxyalkyl, C1-C10 hydroxyalkyl, C1-C10 carboxyalkyl, C1-C10 alkoxylcarbonyl, and C1-C10 alkoxycarbonylalkyl, with the proviso that if E is a single bond, A and B are both —NO— and m and n are both 1, and (i) if R¹, R², R⁴, R⁵, R⁷ and R⁸ are H, then R³ and R⁶ both are not hydrogen, hydroxyl, halo, alkoxyl, phenoxyl, or trifluoromethoxyl and (ii) if R¹, R³-R⁶ and R⁸ are H, then R² and R⁷ both are not methyl, halo, and methoxycarbonyl.

27. The composition of claim 25, wherein:
A and B are —NO—;
C is a double bond;
D is selected from the group consisting of:
single bond, 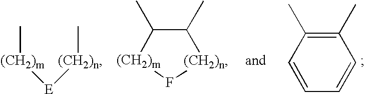
E and F are each independently single bond, double bond, —NR⁹—, or —O—;
m and n are each independently and integer from 0 to 6;
R¹ to R⁸, R¹⁰, and R¹¹ are each independently selected from the group consisting of hydrogen, C1-C10 alkyl, C5-C10 aryl, C1-C10 alkoxyl, amino, hydroxyl, C1-C10 hydroxyalkyl, carboxyl, C1-C10 alkoxylcarbonyl, mono- or polyfluroalkyl, mono- or polyfluroalkoxyl, cyano, nitro, C1-C10 thioalkyl, and C1-C10 sulfonylalkyl; and
R⁹ is selected from the group consisting of hydrogen, C1-C10 alkyl, C3-C10 cycloalkyl, C1-C10 acyl, C5-C10 aryl, carboxyalkyl, C1-C10 alkoxylcarbonyl, and C1-C10 alkoxycarbonylalkyl, with the proviso that if E is a single bond, A and B are both —NO— and m and n are both 1, and (i) if R¹, R², R⁴, R⁵, R⁷ and R⁸ are H, then R³ and R⁶ both are not hydrogen, hydroxyl, alkoxyl, or trifluoromethoxyl and (ii) if R¹, R³-R⁶ and R⁸ are H, then R² and R⁷ both are not methyl.

28. The composition of claim 25, wherein:
A and B are —NO—;
C is a double bond;
D is selected from the group consisting of:
single bond, 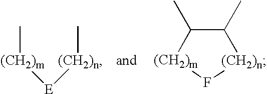
E and F are each independently single bond, double bond, —NR⁹—, or —O—;
m and n are each independently an integer from 0 to 6;
R¹ to R⁸, R¹⁰, and R¹¹ are each independently selected from the group consisting of hydrogen, C1-C10 alkyl, C1-C10 alkoxyl, mono- or polyfluroalkyl, and mono- or polyfluroalkoxyl; and
R⁹ is selected from the group consisting of hydrogen, C1-C10 alkyl, C3-C10 cycloalkyl, C1-C10 acyl, and C5-C10 aryl, and C1-C10 alkoxylcarbonyl, with the proviso that if E is a single bond, A and B are both —NO— and m and n are both 1, and (i) if R¹, R², R⁴, R⁵, R⁷ and R⁸ are H, then R³ and R both are not hydrogen, hydroxyl, alkoxyl, or trifluoromethoxyl and (ii) if R¹, R³-R⁶ and R⁸ are H, then R² and R⁷ both are not methyl.

29. The composition of claim 25, wherein A and B are —SO—; C is a single bond;
D is selected from the group consisting of:
single bond, 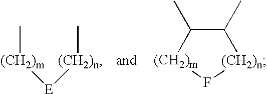
E and F are each independently single bond, double bond, —NR⁹—, or —O—;
m and n are each independently an integer from 0 to 6;
R¹ to R⁸, R¹⁰, and R¹¹ are each independently selected from the group consisting of hydrogen, C1-C10 alkyl, C1-C10 alkoxyl, mono- or polyfluroalkyl, and mono- or polyfluroalkoxyl; and
R⁹ is selected from the group consisting of hydrogen, C1-C10 alkyl, C3-C10 cycloalkyl, C1-C10 acyl, and C5-C10 aryl, and C1-C10 alkoxylcarbonyl.

30. The composition of claim 25, or a pharmaceutically acceptable derivative thereof, wherein at least one of R¹-R⁸ is electron withdrawing substituents.

31. The composition of claim 25, wherein at least two of R¹-R⁸ are electron withdrawing substituents.

32. The composition of claim 25, wherein at least one of R¹-R⁸ is selected from haloalkoxy, alkylsulfonyl and haloamido groups.

33. The composition of claim 25, wherein at least two of R¹-R⁸ are selected from haloalkoxy, alkylsulfonyl and haloamido groups.

34. The composition of claim 25, wherein R¹, R⁴, R⁵ and R⁸ are each hydrogen.

35. The composition of claim 25, wherein, -A-C—B— is —NO═NO—.

36. The composition of claim 25, wherein D is alkylene or a single bond.

37. The composition of claim 25, wherein D is ethylene or a single bond.

38. The composition of claim 25, wherein R² and R⁷ are each independently hydrogen or C1-C10 polyhaloalkylcarbonylamino.

39. The composition of claim 25, wherein R² and R⁷ are each independently hydrogen or trifluoromethylcarbonylamino.

40. The composition of claim 25, wherein R² and R⁷ are each hydrogen.

41. The composition of claim 25, wherein R² and R⁷ are each trifluoromethylcarbonylamino.

42. The composition of claim 25, wherein R³ and R⁶ are each independently hydrogen, polyhaloalkoxy, halo, aralkoxy, alkylsulfonyl or polyhaloalkylheteroaryloxy.

43. The composition of claim 25, wherein R³ and R⁶ are each independently hydrogen, difluoromethoxy, 2,2,2-trifluoroethoxy, 1,1,2-trifluoro-2-chloroethoxy, 1,1,2,3,3,3-hexafluoropropoxy, fluoro, benzyloxy, ethanesulfonyl or 5-trifluoromethyl-2-pyridyloxy.

44. The composition of claim 25, wherein R³ and R⁶ are each independently polyhaloalkoxy, halo, aralkoxy, alkylsulfonyl or polyhaloalkylheteroaryloxy.

45. The composition of claim 25, wherein R³ and R⁶ are each independently difluoromethoxy, 2,2,2-trifluoroethoxy, 1,1,2-trifluoro-2-chloroethoxy, 1,1,2,3,3,3-hexafluoropropoxy, fluoro, benzyloxy, ethanesulfonyl or 5-trifluoromethyl-2-pyridyloxy.

46. The composition of claim 25, wherein the compound has formula: 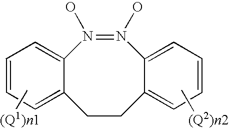
wherein n₁ and n₂ are each independently an integer from 1 to 4 and Q¹ and Q² are each independently selected from haloalkoxy, alkylsulfonyl and haloamido groups, with the proviso that Q¹ and Q² are both not trifluoromethoxyl.

47. The composition of claim 46, wherein the compound has formula:

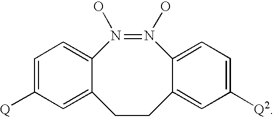

48. A method of treating, preventing, or ameliorating the symptoms of a disease or disorder that is modulated or otherwise affected by Bcl-2 protein or in which Bcl-2 protein is implicated, comprising administering to a subject in need thereof an effective amount of a compound of claim 1.

49. The method of claim 48, wherein the disease or disorder is a Bcl-2 or Bcl-XL mediated disease or disorder.

50. The method of claim 48, wherein the disease or disorder is characterized by overexpression of a Bcl-2 or Bcl-XL protein.

51. A method of treating, preventing, or ameliorating the symptoms of a disease or disorder that is modulated or otherwise affected by Bcl-2 protein or in which Bcl-2 protein is implicated, comprising administering to a subject in need thereof an effective amount of a compound of claim 23.

52. The method of claim 48, wherein the disease or disorder is selected from cancers, tumors, hyperproliferative diseases, acquired immune deficiency syndrome, degenerative conditions, and vascular diseases.

53. The method of claim 52, wherein the cancer is selected from B-cell lymphoma including B-cell lymphoma-2, B-cell leukemia, skin cancer, pancreatic cancer, ovarian cancer, liver cancer, bladder cancer, adrenal carcinoma, breast cancer, prostate cancer and colorectal cancer.

54. A method of modulating the activity of a Bcl-2 protein, comprising administering to a subject in need thereof an effective amount of a compound of claim 1.

55. A method of modulating the activity of a Bcl-2 protein, comprising administering to a subject in need thereof an effective amount of a compound of claim 23.

56. The method of claim 54, wherein the Bcl-2 protein is selected from anti-apoptotic Bcl-2 protein, Bcl-2 and Bcl-X_{L}.

57. A method of antagonizing Bcl-2 protein, comprising administering to a subject in need thereof an effective amount of a compound of claim 1.

58. A method of antagonizing Bcl-2 protein, comprising administering to a subject in need thereof an effective amount of a compound of claim 23.

59. The method of claim 57, wherein the Bcl-2 protein is selected from anti-apoptotic Bcl-2 protein, Bcl-2 and Bcl-X_{L}.

60. A method of altering the interaction of an anti-apoptotic Bcl-2 protein, comprising administering to a subject in need thereof an effective amount of a compound of claim 1.

61. A method of altering the interaction of an anti-apoptotic Bcl-2 protein, comprising administering to a subject in need thereof an effective amount of a compound of claim 23.

62. The method of claim 60, wherein the Bcl-2 protein is selected from anti-apoptotic Bcl-2 protein, Bcl-2 and Bcl-X_{L}.

63. A method of inducing apoptosis, comprising administering to a subject in need thereof an effective amount of a compound of claim 1.

64. A method of inducing apoptosis, comprising administering to a subject in need thereof an effective amount of a compound of claim 23.

65. The compound of claim 1 that is selected from

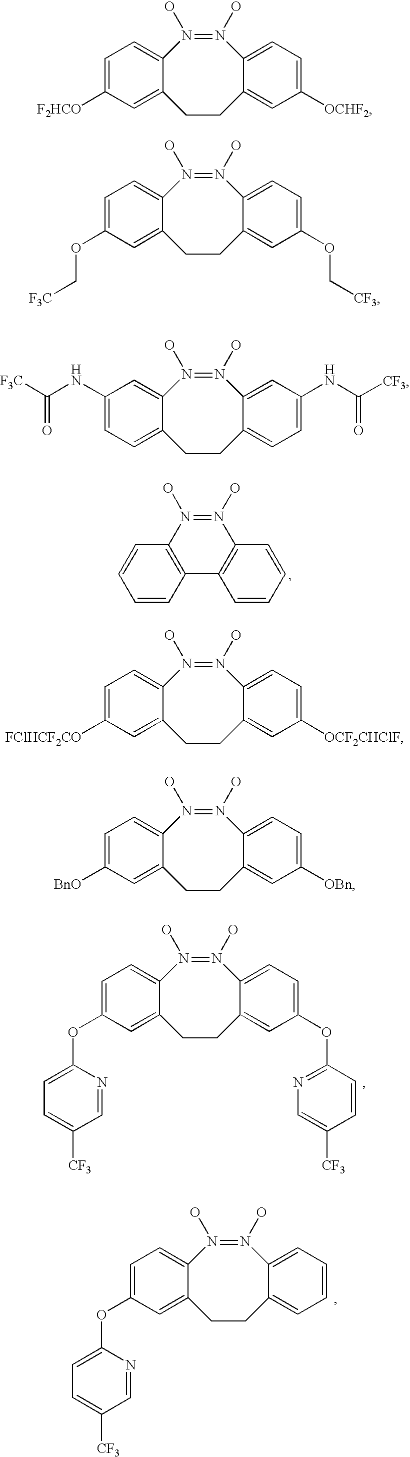

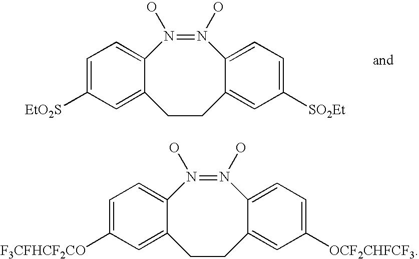

66. The composition of claim 25, wherein the compound is selected from 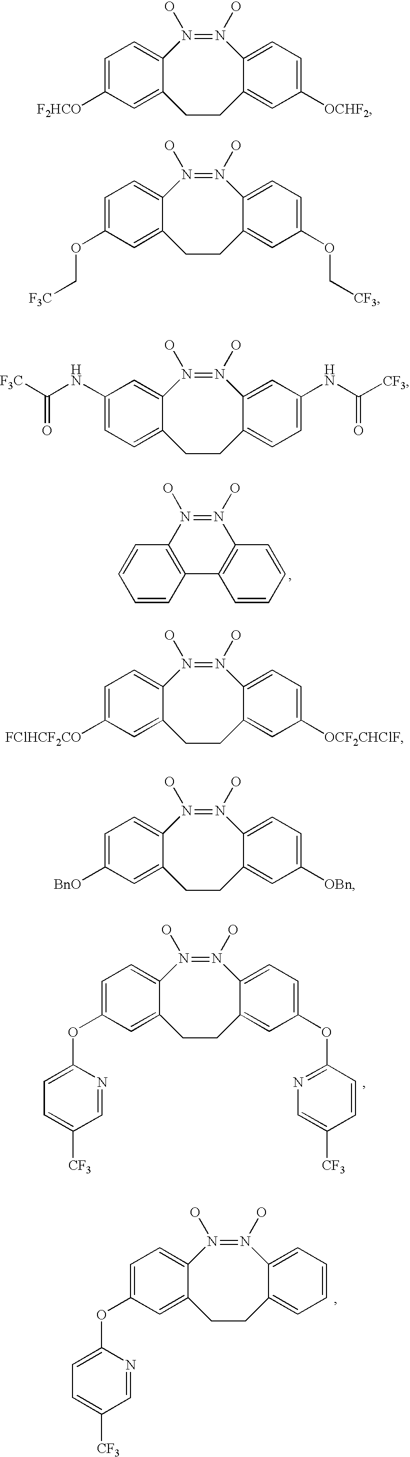 [chemical structure]
